SEQUENCE LISTING (68 sequences)

SEQ ID NO: 1 - DNA, 584 bp, Artificial sequence
  Cauliflower mosaic virus derived 35S promoter
ggccggccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgactagtggtacaattctcaacacaacatatacaaaacaaacgaatctcaagcaatcaagcattctacttctattgcagcaatttaaatcatttcttttaaagcaaaagcaattttctgaaaattttcaccatttacgaacgatagcctagg

SEQ ID NO: 2 - DNA, 141 bp, Tobacco etch virus
ggtacaattctcaacacaacatatacaaaacaaacgaatctcaagcaatcaagcattctacttctattgcagcaatttaaatcatttcttttaaagcaaaagcaattttctgaaaattttcaccatttacgaacgatagcc

SEQ ID NO: 3 - DNA, 795 bp, Artificial sequence
  Neomycin-kanamycin phosphotransferase type II
atgattgaacaggatggtcttcatgctggatctccagctgcatgggttgaacgactctttgggtatgactgggcacaacagaccataggttgctctgatgctgcagttttcaggcttagtgcacaagggagacctgttctcttcgtcaagactgacttgtccggagctttgaatgagcttcaggatgaggctgccagactttcatggcttgctacaactggtgttccttgcgctgcagttcttgacgttgttacagaggctggaagggattggttgcttcttggtgaagtgccaggacaggacttgctttctagtcatctggcaccagctgagaaggtgtctataatggctgatgccatgcgaagacttcacacacttgatcctgccacatgtcctttcgatcaccaggctaagcataggattgagagggctaggactagaatggaagcaggtttggtggaccaagacgatcttgatgaggagcatcaaggacttgctccagctgaacttttcgcaaggctcaaagctaggatgccagatggagaggatcttgttgtgactcacggagatgcttgcttgcccaacatcatggttgagaatgggaggttctctggattcatcgactgtggtagactcggagttgcagataggtaccaggacattgcacttgcaactcgtgacatcgctgaggaacttggaggtgaatgggcagatcgtttccttgtcctctatgggattgctgcacctgattcacagcgtatcgcattctaccgactgctcgatgagttcttctga

SEQ ID NO: 4 - protein, 264 aa, Artificial sequence [sequence omitted]

SEQ ID NO: 5 - DNA, 419 bp, Artificial sequence
  CaMV PolyA Terminator
accggtgaaatcaccagtctctctctacaaatctatctctctctattttctccataataatgtgtgagtagttcccagataagggaattagggttcttatagggtttcgctcacgtgttgagcatataagaaacccttagtatgtatttgtatttgtaaaatacttctatcaataaaatttctaattcctaaaaccaaaatccagtactaaaatccagatcacctaaagtccctatagatctttgtcgtgaatataaaccagacatgagacgactaaacctggagcccagacgccgttcgaagctagaagtaccgcttaggcaggaggccgttagggaaaagatgctaaggcagggttggttacgttgactcccccgtaggtttggtttaaatatgataaagtggacgaattcaagctt

SEQ ID NO: 6 - DNA, 835 bp, Artificial sequence
  35S Promoter from pBI121
agattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacg

SEQ ID NO: 7 - DNA, 75 bp, Artificial sequence
  Plant secretion leader peptide
atggagtccttccgaaggttttctctcctgagtttcattgcgctgttggcctattttgctttcctggctagcgct

SEQ ID NO: 8 - protein, 25 aa, Artificial sequence
  Plant secretion leader peptide
MESFRRFSLLSFIALLAYFAFLASA

SEQ ID NO: 9 - protein, 33 aa, Artificial sequence
  Spider toxin
ECKGFGKSCVPGKNECCSGYACNSRDKWCWVLL

SEQ ID NO: 10 - DNA, 102 bp, Artificial sequence
  Spider toxin coding sequence
gaatgtaaaggattcggcaagtcttgcgttcctggaaagaacgaatgctgtagtggttacgcctgcaattcacgtgacaaatggtgtaaggtccttctctaa

SEQ ID NO: 11 - DNA, 177 bp, Artificial sequence
  Fused Plant secretion leader peptide and Spider toxin coding sequence
atggagtccttccgaaggttttctctcctgagtttcattgcgctgttggcctattttgctttcctggctagcgctgaatgtaaaggattcggcaagtcttgcgttcctggaaagaacgaatgctgtagtggttacgcctgcaattcacgtgacaaatggtgtaaggtccttctctaa

SEQ ID NO: 12 - protein, 58 aa, Artificial sequence
  Fused Plant secretion leader peptide and Spider toxin
MESFRRFSLLSFIALLAYFAFLASAECKGFGKSCVPGKNECCSGYACNSRDKWCKVLL

SEQ ID NO: 13 - DNA, 618 bp, Artificial sequence
  3UTR and terminator of ribosomal protein S40
gcgatcgcgttatttggcctctcgttggatctccttatgtagctttctcaatgtttcattttgttttttcttagtttgaaaagagttttggaattactatgctttgttatagtctcaactctgaacagctttggatctttttctgtatgaccataagatattcaaaaattggtagtcattttatctcacaaaaaaaccttgaatttgtgctgtatgattttgtaaagtcttaggtgccaaatctttgattggagttgataaaatatttttatttggttgtaattttgtaatatcccaggatatttcacaaattgaacatagactacagaattttagaaaacaaactttctctctcttatctcacctttatcttttagagagaaaaagttcgatttccggttgaccggaatgtatctttgttttttttgttttgtaacatatttcgttttccgatttagatcggatctccttttccgttttgtcggaccttcttccggtttatccggatctaataatatccatcttagacttagctaagtttggatctgttttttggttagctcttgtcaatcgcctcatcatcagcaagaaggtgaaatttttgacaaataaatctta

SEQ ID NO: 14 - DNA, 356 bp, Artificial sequence
  UBQ1 60S ribosomal protein L40-2 derived from Arabidopsis thaliana
gaatcatgtagtgtctttggaccttgggaatgatagaaacgatttgttatagctactctatgtatcagaccctgaccaagatccaacaatctcataggttttgtgcatatgaaaccttcgactaacgagaagtggtcttttaatgagagagatatctaaaatgttatcttaaaagcccactcaaatctcaaggcataaggtagaaatgcaaatttggaaagtgggctgggccttttgtggtaaaggcctgtaacctagcccaatattagcaaaaccctagacgcgtacattgacatatataaacccgcctcctccttgtttagggtttctacgtgagagaagacgaaacacaaaag

SEQ ID NO: 15 - protein, 40 aa, Artificial sequence
  Spider Toxin
MGYDIECNENLPCCKHRKLECVETSGYWWYKRKYCRPIKG

SEQ ID NO: 16 - DNA, 120 bp, Artificial sequence
  Spider Toxin coding sequence
atgggatacgatatcgaatgcaacgagaaccttccgtgttgtaaacaccgcaagttggagtgtgtggaaacctcaggctattggtggtacaaacgtaagtattgtaggccgatcaaaggg

SEQ ID NO: 17 - DNA, 204 bp, Artificial sequence
  Fused Plant secretion leader peptideand Spider toxin coding sequence
atggagagctttcgacggttctctctcctgagttttattgcgctgcttgcctactttgccttcttggcttctgctcggtgtatgggatacgatatcgaatgcaacgagaaccttccgtgttgtaaacaccgcaagttggagtgtgtggaaacctcaggctattggtggtacaaacgtaagtattgtaggccgatcaaagggtaa

SEQ ID NO: 18 - protein, 67 aa, Artificial sequence
  Fused Plant secretion leader peptideand Spider toxin
MESFRRFSLLSFIALLAYFAFLASARCMGYDIECNENLPCCKHRKLECVETSGYWWYKRKYCRPIKG

SEQ ID NO: 19 - DNA, 666 bp, Artificial sequence
  AtUBQ1 Terminator
agagactcttatcaagaatcccatctcttgcttgcttttttttgttgtcttccctttgatagggtttgtttttcttgtttcagtgactttctatgttaaaagataatgtcagtaaaaggatttggttttctattattctgaatcgattacggaagattcttgcttaattccaatctatacaagtatcgtgaaataatgaccgtttatgtgattaggagacgtgtttcattaataaaatataagatcaatacattgttagtagtgataaactatgtacaaattgtattgattgtaaaagaaacacaataggttccttttttctacaatatattgtgacagactctctgttttaacgaatgaattaaatttgtcaacgaaaaaattaaagaatgatgtgatcactttgcaccttggttcatcttcagaggcttctgcatgttccatgcattgaggctctctacggtcacaggttgtgagccgttgttaaagacaaacaaatgagctttttctcctactgcttttgtcggatacactctaagatgttatcactgtctttccttttgctccaaagctctccacaaccgagtgatcaatctgcaaattcactcaattatgttacctatttgcttctctatacaagagtaatattcagtaaaatgagtcgac

SEQ ID NO: 20 - DNA, 1103 bp, Artificial sequence
  Arabidopsis thaliana derived Actin 7 promoter and 5 UTR sequence
tagataaacaatacactttacacacattattttcttagaattgggtctcatatagaacactcacaaaggtgacttataaaaaatgcttcaaagatcctagtcttttagtgtgcattcctcaaaatcgtagttgatatgtatcttgctcctattcgggttttggtcttagaattttggtttcaggaattttcaattaactcattaaaacatttataacataaaaagatatatcacgaaaaccgatcaatcatttaatatatttttattttattttattttttgtcacaataaaatcctagacaatagtgtttaaacatttatcaaaaaattcaccttacgattaatttgttttatattttgtgatgatgcaatttaatctatataaattataaaacatatcgataatctgtgtttttctttttctacatataaaattcataaaaatgagagttttgaagctcttaaaatggactagtcaacaattggccaatctttgttctaaattgctaataaacgaccatttccgtcaattctccttggttgcaacagtctacccgtcaaatgtttactaatttataagtgtgaagtttgaattatgaaagacgaaatcgtattaaaaattcacaagaataaacaactccatagattttcaaaaaaacagtcacgagaaaaaaaccacagtccgtttgtctgctcttctagtttttattatttttctattaatagttttttgttatttcgagaataaaatttgaacgatgtccgaaccacaaaagccgagccgataaatcctaagccgagcctaactttagccgtaaccatcagtcacggctcctcgggctaattcatttgaaccgaatcataatcaacggtttagatcaaactcaaaacaatctaacggcaacatagacgcgtcggtgagctaaaaagagtgtgaaagccaggtcaccatagcattgtctctcccagattttttatttgggaaataatagaagaaatagaaaaaaataaaagagtgagaaaaatcgtagagctatatattcgcacctgtactcgtttcgctttccttagtgttagctgctgccgctgttgtttctcctcca

SEQ ID NO: 21 - DNA, 75 bp, Artificial sequence
  Plant secretion leader peptide coding sequence
atgggtctccagcaagggttggttacctggtttgttggcgtcctgttcctgagcactttgcttctctccaacgct

SEQ ID NO: 22 - protein, 25 aa, Artificial sequence
  Plant secretion leader peptide
MGLQQGLVTWFVGVLFLSTLLLSNA

SEQ ID NO: 23 - DNA, 111 bp, Artificial sequence
  Spider Toxin P11060 coding sequence
gcctgtgtcggagaaaaccagcaatgcgcggattgggcaggacctcattgttgcgacggctactactgcacgtgtaggtatttcccgaagtgcatctgccggaacaacaat

SEQ ID NO: 24 - protein, 37 aa, Artificial sequence
  Spider Toxin P11060
ACVGENQQCADWAGPHCCDGYYCTCRYFPKCICRNNN

SEQ ID NO: 25 - DNA, 189 bp, Artificial sequence
  Fused Plant secretion leader peptide and Spider Toxin P11060 coding sequence
atgggtctccagcaagggttggttacctggtttgttggcgtcctgttcctgagcactttgcttctctccaacgctgcctgtgtcggagaaaaccagcaatgcgcggattgggcaggacctcattgttgcgacggctactactgcacgtgtaggtatttcccgaagtgcatctgccggaacaacaattaa

SEQ ID NO: 26 - protein, 62 aa, Artificial sequence
  Fused Plant secretion leader peptide and Spider Toxin P11060
MGLQQGLVTWFVGVLFLSTLLLSNAACVGENQQCADWAGPHCCDGYYCTCRYFPKCICRNNN

SEQ ID NO: 27 - DNA, 924 bp, Artificial sequence
  Arabidopsis thaliana derived Actin 7 gene 3′ UTR and terminator
gtgtgtcttgtcttatctggttcgtggtggtgagtttgttacaaaaaaatctattttccctagttgagatgggaattgaactatctgttgttatgtggattttattttcttttttctctttagaaccttatggttgtgtcaagaagtcttgtgtactttagttttatatctctgttttatctcttctattttctttaggatgcttgtgatgatgctgtttttttttgtccctaagcaaaaaaatatcatattatatttggtccttggttcatttttttggtttttttttgtcttcacatataaatattgtttgaatgtcttcaatcttttatttgtatgagacaattatttaagtatcgggtgacaatgcagctattatgtattgtcgattgttatattggcgcccaaaatatatacttagcctaagaatttggtaagtgagtggcttatgttttactccagcaaaaattgtgtgtgtattaccattctgatgcgaaacaagaaaagaatttgatctaagaaaccaagtttattcactagttaaaaaacaaatgacctaatgtaatcgactccacatatcaaaatacgtaaaacaaacattgtatgttgacaaaagggaaaagaaatgatttatttggttaaaaagaaagctggattcaattgcaacagtttagtcgaaatcattttgaaaggcttacaatggattgaatgtgaatattccattaagccgcttctgtctacacagaatgttacgcttggagagcagcaatcattttcacgtttttatctttttaggtggacatgtatattattggttacgcctttggagtttttcgaaatttatttctttcaaatcacaagatgactaaacatcacaatctgtttatcttcctaactagttaaatttttgtccccaccattggc

SEQ ID NO: 28 - DNA, 998 bp, Artificial sequence
  Arabidopsis thaliana derived Delta-TIP gene Promoter and 5 UTR, DELTA-TIP aquaporin TIP2-1
gcgcctaaataatagagggacccaaaataacattgttgaaatggaatataatcataaatgagtcacatcttcaaaatctgatcaaatcacttggaaaaaaaaagatcaagtcacatgcaaccagtaaccacaatcatattttggccggtctatcaccatacacattgtttacagctcacacttgtccctccttatatttgttcgtatccatttgttttcttctctctctactctaatctcatctttatgtttatccctttttgaattttgaagctactctgtcctttgggtccaccctccaccacctctaatttcaatcacgttaaaccggccatattactttaaaccgaccgttttctactaataggaaaatccgacccaaatttttgactaattataagacataccgccgctataccatgatattgtcaaagaaaaagtttctaaccaataaccactatagactaaaagatttcaaataatttgattgatactgcattgaacattctacaattctacttatattgtgtagttcaaataatgagaacatatcggaaagactagccgctattagattatgaatgcgtatgaatacattatacattatagtatatatatatatatatacacatatattaaaatgtattactatataataatttgtcttagtatagtactcaaagaaatagagttttcttacgtagatgtcatatattttagtcattttaggcaccttcctaactcaaatcacgaatagaacttatgtagcaaaatgtaaacacatgaaatcatttattaattcgggtccaacgtaacccaccacaccacagaaactcattaaatagtatttttttataaaacttaagttatattcctggatacaggaacgatactcctatataaagagaacagcattcaaaaggtcttatcatcttcttcactaaacaaaaaaaaacccttcaaaacatttccttattctttcttcttcatctacaaca

SEQ ID NO: 29 - DNA, 141 bp, Artificial sequence
  Spider Toxin P61095 coding sequence
aaggaatgtatgactgacggtacggtgtgttacatccacaatcacaacgattgttgcggatcctgtctctgctcaaatggacctattgcaaggccatgggaaatgatggtcggcaactgtatgtgtggtcccaaagcgtaa

SEQ ID NO: 30 - protein, 46 aa, Artificial sequence
  Spider Toxin P61095
KECMTDGTVCYIHNHNDCCGSCLCSNGPIARPWEMMVGNCMCGPKA

SEQ ID NO: 31 - DNA, 216 bp, Artificial sequence
  Fused Plant secretion leader peptide and Spider toxin P61095 coding sequence
atggagtctttcagacggttttcgctccttagcttcatcgctcttctggcctatttcgctttcttggcttccgcaaaggaatgtatgactgacggtacggtgtgttacatccacaatcacaacgattgttgcggatcctgtctctgctcaaatggacctattgcaaggccatgggaaatgatggtcggcaactgtatgtgtggtcccaaagcgtaa

SEQ ID NO: 32 - protein, 71 aa, Artificial sequence
  Fused Plant secretion leader peptide and Spider toxin P61095
MESFRRFSLLSFIALLAYFAFLASAKECMTDGTVCYIHNHNDCCGSCLCSNGPIARPWEMMVGNCMCGPKA

SEQ ID NO: 33 - DNA, 764 bp, Artificial sequence
  Arabidopsis thaliana derived Delta-TIP gene (At3g16240) 3′ UTR and Terminator, DELTA-TIP aquaporin TIP2-1
ctcgagggaaacaagtgatgattcttgattcatgtttctgtgtttggttactttgcctcgatctttcttgttttgtttggagtttggtccggtttcgttgtaatttttatccaatttgtatgaatattatttaatggatggctgttttggttataaattaactaatggatgtatgaatatatagtttcacctaaataggtagttgtaatgttgtatagctaattatcgatccatatatataaaaaatagatcgttacaattttatgatatagtaacaactaacaacaatgacaaattaatggatggtttcaagtagaagtttcgagatcatcgtcattaggtatattcttagggatgttccactcatgagctttccactctggcaattgttgtatgacaacctatgcaataatttattaatatcaataaatcacatgcatgtatagaaaaaaatgaagattatatattatagacagagaaaaagagaggttatatattattaccaatccggtagaatctggatttcccacgttggtttgacaagctagtctccaattttttggtttctgtgaaaacattattcgattttactcccataaccaagatgatacaagattactaaatactataggataatttcttaaataaaactatacccttttgagtttctccttctcaatatcagttcgcggatttagaagctcctttccatttacaatctgaaaagaaatatccatcaatatctcataaaccatcataagctt

SEQ ID NO: 34 - DNA, 937 bp, Artificial sequence
  Arabidopsis thaliana derived Gamma-TIP2 (At3g26520) Promoter and 5UTR, TIP2 aquaporin TIP1-2
cgttggaagctgtatatcctctgacgccgcattaaatgcgtctgatacgtatctccatctctctttgctcgcgctttttattgtcacatccgggaatttttctaataattttctcttatgttttctttccaagttgctccgatatgattattgtgattttaaaacatcacatgttgtttccgtagtacttgtgctgtttaccaaatcgtagtacttctgctggtcatagattcaaggtaaaagcccattcaccttgttgaataatcaatccagtacaatactcacccgaaaaagttaccagcccaatatcgtggtatatactccatttcaatactaatttttttgttttttaatgattatttttttaaaaaaaaaatgaaataaatcctttcatagtacatgctgctgcagttgtaaattgcatgtattttatgaaacgttcagtacaaacttagaaagttgtagtaatgaaagctctaaattgagatctacactcataaatagttcatgctttatttagtagtaactgcataagtaatgcatgctttttttttttgatacgttgtaatcattgccctacaatctctagctcccctacatctacaagtctgctgtcgggaatatggttttgccgcccatccgtagaggctatatcggatatttcgcatggtaaaaaaacgttatatacccaacttttctgggagaaggtcattctcttaataaggatgtcaaatttggtcattcagtaaattaacccttaaaaaaatgcaaaataatttgtttatgtgacaaagaaatggattttatctataactatcaagaagaagaaaacactcggctatatatatacgtacaacacataagaaaaacaaaccaatcacttcactctctctaatcaaaaaggcttttaacctcagccgttaaatctttctccgat

SEQ ID NO: 35 - DNA, 981 bp, Beauveria bassiana
tcgcctctcgctcccagagcgggcacttgtgccacaaagggtcgcccagccggtaaagtcctccagggatattgggaaaactgggatggagcaaagaacggggttcatccgcccttcggttggacgcctattcaaaatcctgacatccgtaagcacgggtacaacgttatcaacgctgccttcccaattatccagcctgatggaactgcactgtgggaggatgggatggatactggagtgaaagttgcctcgcccgcagacatgtgcgaggcaaaggcggctggagcaaccattcttatgtctatcgggggtgctacggcggcaattgatttgtcatctagtgccgttgccgacaaattcgtttcgacaatagtcccaatcctcaaaaagtacaacttcgacggaatagacatagatatcgaatccggtctgacagggtccgggaatatcaatacgctgagcacgtctcagacaaacctcatcaggattatcgacggcgtgttggctcagatgccggcaaacttcggtctgactatggctccggagactgcttatgttaccggaggaacgattacctacgggagcatttggggttcatacctgcctatcataaagaaatatctggataacgggcgactttggtggctcaatatgcaatactataacggggaaatgtacggctgtagcggcgatagtcataaggccggcacagtggagggattcatagcacagacagattgcctgaataagggcctgagtattcagggtgtgaccatcaccatcccctacgacaaacaagttcctggattgccggctcaaccaggggcaggtggcggtcacatgtcacctagcaacgtggcacaagttctctcccactataaaggcgctctcaagggactgatgacttggtcactcaattgggacggctcgaagaattggaccttcggcgataatgtcaagggaaccttgggcaccgcctaa

SEQ ID NO: 36 - protein, 326 aa, Beauveria bassiana
SPLAPRAGTCATKGRPAGKVLQGYWENWDGAKNGVHPPFGWTPIQNPDIRKHGYNVINAAFPIIQPDGTALWEDGMDTGVKVASPADMCEAKAAGATILMSIGGATAAIDLSSSAVADKFVSTIVPILKKYNFDGIDIDIESGLTGSGNINTLSTSQTNLIRIIDGVLAQMPANFGLTMAPETAYVTGGTITYGSIWGSYLPIIKKYLDNGRLWWLNMQYYNGEMYGCSGDSHKAGTVEGFIAQTDCLNKGLSIQGVTITIPYDKQVPGLPAQPGAGGGHMSPSNVAQVLSHYKGALKGLMTWSLNWDGSKNWTFGDNVKGTLGTA

SEQ ID NO: 37 - DNA, 1056 bp, Artificial sequence
  Fused Plant secretion leader peptide and Beauveria bassiana chitinase coding sequence
atggagagctttcgacggttttccctcttgtcctttattgccctgttggcgtattttgccttcttggcttctgcttcgcctctcgctcccagagcgggcacttgtgccacaaagggtcgcccagccggtaaagtcctccagggatattgggaaaactgggatggagcaaagaacggggttcatccgcccttcggttggacgcctattcaaaatcctgacatccgtaagcacgggtacaacgttatcaacgctgccttcccaattatccagcctgatggaactgcactgtgggaggatgggatggatactggagtgaaagttgcctcgcccgcagacatgtgcgaggcaaaggcggctggagcaaccattcttatgtctatcgggggtgctacggcggcaattgatttgtcatctagtgccgttgccgacaaattcgtttcgacaatagtcccaatcctcaaaaagtacaacttcgacggaatagacatagatatcgaatccggtctgacagggtccgggaatatcaatacgctgagcacgtctcagacaaacctcatcaggattatcgacggcgtgttggctcagatgccggcaaacttcggtctgactatggctccggagactgcttatgttaccggaggaacgattacctacgggagcatttggggttcatacctgcctatcataaagaaatatctggataacgggcgactttggtggctcaatatgcaatactataacggggaaatgtacggctgtagcggcgatagtcataaggccggcacagtggagggattcatagcacagacagattgcctgaataagggcctgagtattcagggtgtgaccatcaccatcccctacgacaaacaagttcctggattgccggctcaaccaggggcaggtggcggtcacatgtcacctagcaacgtggcacaagttctctcccactataaaggcgctctcaagggactgatgacttggtcactcaattgggacggctcgaagaattggaccttcggcgataatgtcaagggaaccttgggcaccgcctaa

SEQ ID NO: 38 - protein, 351 aa, Artificial sequence
  Fused Plant secretion leader peptide and Beauveria bassiana chitinase
MESFRRFSLLSFIALLAYFAFLASASPLAPRAGTCATKGRPAGKVLQGYWENWDGAKNGVHPPFGWTPIQNPDIRKHGYNVINAAFPIIQPDGTALWEDGMDTGVKVASPADMCEAKAAGATILMSIGGATAAIDLSSSAVADKFVSTIVPILKKYNFDGIDIDIESGLTGSGNINTLSTSQTNLIRIIDGVLAQMPANFGLTMAPETAYVTGGTITYGSIWGSYLPIIKKYLDNGRLWWLNMQYYNGEMYGCSGDSHKAGTVEGFIAQTDCLNKGLSIQGVTITIPYDKQVPGLPAQPGAGGGHMSPSNVAQVLSHYKGALKGLMTWSLNWDGSKNWTFGDNVKGTLGTA

SEQ ID NO: 39 - DNA, 841 bp, Artificial sequence
  Arabidopsis thaliana derived Gamma-TIP2 3′UTR and terminator
agacgtcaaattcaacgttgttaatctgatgaatttttcgtgatttgcttttttaatttgcatcgtaatgggtttctggccgttggatcatttttagatgaatctttgtctattgattgatcatttatgtgtctttgggtttgttgttggagttgtaatatctctggaaggcttttgtgcaattgtattgtacggtttaataactataattgagaaaaccagggaatgtgaattatgaaaatgaatttttttgttattgtttattttacttaaagagccctaactaatttagatcataatttacgagacatggaaattttaatgaataataaacattgtgattgatatttttggttttcatttccgttttttatattttatatcaacacactacagtagtacatatttacacatgatttatgtccaggctaaaattgtatattaagcgttgaaaaatcatcgagtgttacacttaggaggggttcgacttccatgtgtaaaaataatactagcaattcgcaaataactttacaaacacaagtcatgactcatgagctaaggttcaaattactttgggttgcaatgtatgcgtttcatttctatatcgttccaatcacagcattattatttccttctttgtgagtcttcttccaacatttcggcgggaatctgtagggagattttcgtcattcggatttcattttgtttggtttcaaaacgtgaatagcaatagatggatgggaatagattcgctaaaatctttgtggggttaatggataataaagttcgtgaaacgtctggacagtatacgaaccatctacctctataagggcccaagctt

SEQ ID NO: 40 - DNA, 301 bp, Figwort mosaic virus
tttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatgatttcaaaaaacgcagctataaaagaagccctccagcttcaaagttttcatcaacacaaattctaaaaacaaaattttttagagagggggagtg

SEQ ID NO: 41 - DNA, 1212 bp, Beauveria bassiana
ctcatcccaccgatagttactgagattggcgtcgagaagcggagcggtggctttgctaactccgtgtacttcactaattggggtatttacggtcgaaacttccaaccgaccgatttgcctgcgagcaagatcactcacgtgatatacgccttcatgaacgtcacatcaaatgggactgtctattctggggatagctatgctgactatgagaaacattatgctaacgatacatggaaggacgacggtaacaacgcctacgggtgtgtgaagcagctgttccttctcaaaaagcagcaccgtcacttgaaagtgatgctgtcgataggtggttggacctggtccaaaaatttccccgcaacagcttcaagcgcttctaccagaaaaacgttcgctcagagtgctgtgggactcatgaaagactggggatttgatggaatcgatattgactgggagtacccggaagacgcaacccaggctcaagacatgatcttcttgttgcaagctgttagggacgagcttgatagctacgcttcgcagcacgctaagaaccatcacttcttgctgtcaatcgcagcgccaacaggtgccaaggaatacaagaaacttaagttggcggagcttgggcaagtcctcgattacatcaatctgatggcctacgacttcgcaggggcgtggtccaatgccaccgggcatggagccaatctgtacaacaatacggagaacccagcagccacaccctttaacacgaaggatgcagtggacgcatatatcaatggaggcgtccctgcgaacaaactcgtcctcggaatgccgctctatggccgtagcttccagcaaaccgaaggaattgggaaaccttacacgggtgtcggtagtggcacctgggaatatgggacatgggattatcgcgtgctccctaaacctggcgcagaaatcatgtgtgataacaccgcccagggatgctactcctatgatgcccagactaaagagttgatctcgtttgatacccccgatattgtggccgcgaaggtggaatggcttaaggagaaaggtcttgggggatcaatgttttgggaggcgagtggagataagaacggctctgactccctgattaacaagtccttcgttggcctgggaagtctggaatcaacgccaaacttcttggactatcccaatagcacgtacgctaacatcattaagggcatggcctaa

SEQ ID NO: 42 - protein, 403 aa, Beauveria bassiana
LIPPIVTEIGVEKRSGGFANSVYFTNWGIYGRNFQPTDLPASKITHVIYAFMNVTSNGTVYSGDSYADYEKHYANDTWKDDGNNAYGCVKQLFLLKKQHRHLKVMLSIGGWTWSKNFPATASSASTRKTFAQSAVGLMKDWGFDGIDIDWEYPEDATQAQDMIFLLQAVRDELDSYASQHAKNHHFLLSIAAPTGAKEYKKLKLAELGQVLDYINLMAYDFAGAWSNATGHGANLYNNTENPAATPFNTKDAVDAYINGGVPANKLVLGMPLYGRSFQQTEGIGKPYTGVGSGTWEYGTWDYRVLPKPGAEIMCDNTAQGCYSYDAQTKELISFDTPDIVAAKVEWLKEKGLGGSMFWEASGDKNGSDSLINKSFVGLGSLESTPNFLDYPNSTYANIIKGMA

SEQ ID NO: 43 - DNA, 1287 bp, Artificial sequence
  Fused Plant secretion leader peptide and Beauveria bassiana endochitinase  coding sequence
atgggccttcaacagggcttggttacttggtttgttggcgtcctgtttctctctactctccttttgtcgaacgcactcatcccaccgatagttactgagattggcgtcgagaagcggagcggtggctttgctaactccgtgtacttcactaattggggtatttacggtcgaaacttccaaccgaccgatttgcctgcgagcaagatcactcacgtgatatacgccttcatgaacgtcacatcaaatgggactgtctattctggggatagctatgctgactatgagaaacattatgctaacgatacatggaaggacgacggtaacaacgcctacgggtgtgtgaagcagctgttccttctcaaaaagcagcaccgtcacttgaaagtgatgctgtcgataggtggttggacctggtccaaaaatttccccgcaacagcttcaagcgcttctaccagaaaaacgttcgctcagagtgctgtgggactcatgaaagactggggatttgatggaatcgatattgactgggagtacccggaagacgcaacccaggctcaagacatgatcttcttgttgcaagctgttagggacgagcttgatagctacgcttcgcagcacgctaagaaccatcacttcttgctgtcaatcgcagcgccaacaggtgccaaggaatacaagaaacttaagttggcggagcttgggcaagtcctcgattacatcaatctgatggcctacgacttcgcaggggcgtggtccaatgccaccgggcatggagccaatctgtacaacaatacggagaacccagcagccacaccctttaacacgaaggatgcagtggacgcatatatcaatggaggcgtccctgcgaacaaactcgtcctcggaatgccgctctatggccgtagcttccagcaaaccgaaggaattgggaaaccttacacgggtgtcggtagtggcacctgggaatatgggacatgggattatcgcgtgctccctaaacctggcgcagaaatcatgtgtgataacaccgcccagggatgctactcctatgatgcccagactaaagagttgatctcgtttgatacccccgatattgtggccgcgaaggtggaatggcttaaggagaaaggtcttgggggatcaatgttttgggaggcgagtggagataagaacggctctgactccctgattaacaagtccttcgttggcctgggaagtctggaatcaacgccaaacttcttggactatcccaatagcacgtacgctaacatcattaagggcatggcctaa

SEQ ID NO: 44 - protein, 428 aa, Artificial sequence
  Fused Plant secretion leader peptide and Beauveria bassiana endochitinase
MGLQQGLVTWFVGVLFLSTLLLSNALIPPIVTEIGVEKRSGGFANSVYFTNWGIYGRNFQPTDLPASKITHVIYAFMNVTSNGTVYSGDSYADYEKHYANDTWKDDGNNAYGCVKQLFLLKKQHRHLKVMLSIGGWTWSKNFPATASSASTRKTFAQSAVGLMKDWGFDGIDIDWEYPEDATQAQDMIFLLQAVRDELDSYASQHAKNHHFLLSIAAPTGAKEYKKLKLAELGQVLDYINLMAYDFAGAWSNATGHGANLYNNTENPAATPFNTKDAVDAYINGGVPANKLVLGMPLYGRSFQQTEGIGKPYTGVGSGTWEYGTWDYRVLPKPGAEIMCDNTAQGCYSYDAQTKELISFDTPDIVAAKVEWLKEKGLGGSMFWEASGDKNGSDSLINKSFVGLGSLESTPNFLDYPNSTYANIIKGMA

SEQ ID NO: 45 - DNA, 253 bp, Artificial sequence
  NOS terminator
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 46 - protein, 108 aa, Artificial sequence
  Snowdrop derived GNA - Chain A, Mannose- Specific Agglutinin (Lectin)
AAADNILYSGETLSTGEFLNYGSFVFIMQEDCNLVLYDVDKPIWATNTGGLSRSCFLSMQTDGNLVVYNPSNKPIWASNTGGQNGNYVCILQKDRNVVIYGTDRWATG

SEQ ID NO: 47 - DNA, 501 bp, Artificial sequence
  Fused Plant secretion leader peptide,  Spider toxin P83591 and GNA coding sequence
atggagagctttcgacgcttttccctcttgagcttcatcgccttgctcgcgtatttcgctttcttggcctccgctgaatgtaaaggcttcggaaagtcatgtgtccctggcaagaacgaatgttgtagtggctatgcatgcaattctcgggacaaatggtgtaaagtccttctcgcagctgccgataatatcctgtactcgggtgagacactttctactggggagttcctcaactacgggtcctttgtgtttataatgcaagaggactgcaatcttgttctgtacgatgtcgataagccaatttgggctaccaacactggtggattgagtaggagctgcttcctctcaatgcaaacagacgggaatcttgtggtctacaatccctcgaacaagccgatatgggctagtaatactggcggtcagaacgggaactacgtttgcattctgcaaaaggaccgtaacgtggttatctacggaactgatcggtgggcaacgggttaa

SEQ ID NO: 48 - protein, 166 aa, Artificial sequence
  Fused Plant secretion leader peptide,  Spider toxin P83591 and GNA coding sequence
MESFRRFSLLSFIALLAYFAFLASAECKGFGKSCVPGKNECCSGYACNSRDKWCKVLLAAADNILYSGETLSTGEFLNYGSFVFIMQEDCNLVLYDVDKPIWATNTGGLSRSCFLSMQTDGNLVVYNPSNKPIWASNTGGQNGNYVCILQKDRNVVIYGTDRWATG

SEQ ID NO: 49 - DNA, 528 bp, Artificial sequence
  Fused Plant secretion leader peptide,  Spider toxin P83558 and GNA coding sequence
atggagtctttcagacgcttctccctcttgagttttattgcgctgcttgcctatttcgcctttcttgcttctgctcggtgtatgggttacgatatcgaatgcaacgagaacctcccttgctgtaaacaccgaaagttggagtgcgtggaaacctcaggctattggtggtacaaacgtaagtactgtagaccaatcaaaggggctgctgccgataacatactgtatagcggggaaaccttgtcaactggggagttcctcaactacggaagttttgtcttcatcatgcaagaggattgcaaccttgtgctctacgacgttgacaaaccgatttgggcaacaaatacgggaggtctttctcgatcctgctttctgtctatgcaaactgacggaaatctggtggtttataacccctcgaataagcctatctgggcatcgaataccggcggtcagaatggaaactacgtctgtattctccaaaaggatcggaatgtggtgatatacggcactgatagatgggcgacaggctaa

SEQ ID NO: 50 - protein, 175 aa, Artificial sequence
  Fused Plant secretion leader peptide,  Spider toxin P83558 and GNA coding sequence
MESFRRFSLLSFIALLAYFAFLASARCMGYDIECNENLPCCKHRKLECVETSGYWWYKRKYCRPIKGAAADNILYSGETLSTGEFLNYGSFVFIMQEDCNLVLYDVDKPIWATNTGGLSRSCFLSMQTDGNLVVYNPSNKPIWASNTGGQNGNYVCILQKDRNVVIYGTDRWATG

SEQ ID NO: 51 - DNA, 513 bp, Artificial sequence
  Fused Plant secretion leader peptide,  Spider toxin P11060 and GNA coding sequence
atgggactgcagcaaggcttggttacatggtttgttggcgtcctgtttctctctactctccttttgagcaacgccgcttgtgtcggcgaaaatcagcaatgcgccgattgggcaggtcctcattgctgtgacggttactattgcacctgtcgctatttccccaagtgcatatgccgcaataacaacgccgctgcggataacattctctattccggagaaacgttgagcacgggggagttcctcaattacgggtccttcgtcttcattatgcaagaggactgcaacctggtcctttacgacgttgataagccaatctgggcaactaataccggagggcttagtcgatcgtgtttcctttcaatgcaaacagacggtaacctggtcgtgtacaatccgagcaacaaacctatctgggctagcaataccggcggacagaatggcaattacgtgtgtatcctccagaaggatcggaacgtggttatctacgggaccgatagatgggcaactggataa

SEQ ID NO: 52 - protein, 170 aa, Artificial sequence
  Fused Plant secretion leader peptide,  Spider toxin P11060 and GNA
MGLQQGLVTWFVGVLFLSTLLLSNAACVGENQQCADWAGPHCCDGYYCTCRYFPKCICRNNNAAADNILYSGETLSTGEFLNYGSFVFIMQEDCNLVLYDVDKPIWATNTGGLSRSCFLSMQTDGNLVVYNPSNKPIWASNTGGQNGNYVCILQKDRNVVIYGTDRWATG

SEQ ID NO: 53 - DNA, 540 bp, Artificial sequence
  Fused Plant secretion leader peptide,  Spider toxin P61095 and GNA coding sequence
atggagagctttcgacggttctccctcttgtcttttatagccctgctcgcctatttcgctttcttggcttcggctaaagaatgtatgaccgacgggaccgtgtgttatatccacaatcataacgattgctgtggctcctgcctgtgttcaaatggtcctattgcaagaccttgggagatgatggtgggaaactgtatgtgtggacctaaagccgcagctgccgataacatcctgtatagtggcgaaacactcagcaccggagaatttctgaactacgggagcttcgtctttatcatgcaagaggactgcaacctcgttctttacgacgtcgataagccgatttgggcgacaaacactggaggactttcacgttcttgcttcctctcgatgcaaacggacggtaatttggtcgtgtacaatccctccaataagccaatttgggcaagcaataccggaggtcagaacggtaactacgtttgcatcctccaaaaggatcgcaacgtggttatatacgggactgatcgatgggcaacgggttaa

SEQ ID NO: 54 - protein, 179 aa, Artificial sequence
  Fused Plant secretion leader peptide,  Spider toxin P61095 and GNA
MESFRRFSLLSFIALLAYFAFLASAKECMTDGTVCYIHNHNDCCGSCLCSNGPIARPWEMMVGNCMCGPKAAAADNILYSGETLSTGEFLNYGSFVFIMQEDCNLVLYDVDKPIWATNTGGLSRSCFLSMQTDGNLVVYNPSNKPIWASNTGGQNGNYVCILQKDRNVVIYGTDRWATG

SEQ ID NO: 55 - protein, 121 aa, Artificial sequence
  Mu-hexatoxin-Mg1a
MMTLSPFLLLLIAAVVIGNASEGEVKNEFEERLKDEFKDPSRSEVAEVILLRELEVLEETLFGKEMTSDTEENRNSREKRCMGYDIECNENLPCCKHRKLECVETSGYWWYKRKYCRPIKG

SEQ ID NO: 56 - protein, 82 aa, Artificial sequence
  Delta-ctenitoxin-Pn1a
MKVAIVFLSLLVLAFASESIEENREEFPVEESARCGDINAACKEDCDCCGYTTACDCYWSKSCKCREAAIVIYTAPKKKLTC

SEQ ID NO: 57 - protein, 37 aa, Artificial sequence
  Delta-amaurobitoxin-Pl1d
ACATKNQRCASWAGPYCCDGFYCSCRSYPGCMCRPNS

SEQ ID NO: 58 - protein, 351 aa, Nepenthes khasiana
MNAPCFCFHAQKMRNHKHSTMRGWVVLLLLNLPFLSAFQCGQQAGGALCHSGLCCSQWGWCGTTSDYCGNGCQSQCGGTATTPPPSPPSPPPPATPSPPSPPSPVGGDVSSIITREIFEEMLLHRNNAACPARGFYTYEAFITAARFFSGFGTTGDFNTRKRELAAFLGQTSHETTGGWATAPDGPYAWGYCFKEEVGQPGSYCVPSTQWPCAAGKSYYGRGPIQLSYNYNYGPSGQAIGQPLLENPDLVAGDVIVSFETAIWFWMTPQYDKPSCHDVMIGKWTPSAPDIAAGRFPGYGVTTNIINGGLECGRGPDARVASRIGFYERYCDILGVDYGDNLDCYTQWPFGG

SEQ ID NO: 59 - protein, 318 aa, Nepenthes khasiana
MEIASAKIFFGLSLLGLLALGSAEQCGSQAGGAVCPGGLCCSQYGWCGTTDDYCGAGCQSQCSFSGGDPSSLVTRDKFNQMLKHRNDGGCPAKGFYTYDAFIAAAKSFPAFAATGDAATRKREIAAFLAQTSHETTGGWASAPDGPYAWGYCYLREQGNPGSYCVQSAQWPCVAGKKYYGRGPIQISYNFNYGAAGKAIGVDLLNNPDLVEKDPVVSFKTAIWFWMTPQSPKPSCHAVITGRWTPSAADKSAGRVPGFGVVTNIINGGVECGHGQDARVADRIGFYKRYCDILGVGYGNNLDCYNQRPFGNGLLWATE

SEQ ID NO: 60 - protein, 348 aa, Beauveria bassiana
MAPFLQTSLALLPLLASTMVSASPLAPRAGTCATKGRPAGKVLQGYWENWDGAKNGVHPPFGWTPIQNPDIRKHGYNVINAAFPIIQPDGTALWEDGMDTGVKVASPADMCEAKAAGATILMSIGGATAAIDLSSSAVADKFVSTIVPILKKYNFDGIDIDIESGLTGSGNINTLSTSQTNLIRIIDGVLAQMPANFGLTMAPETAYVTGGTITYGSIWGSYLPIIKKYLDNGRLWWLNMQYYNGEMYGCSGDSHKAGTVEGFIAQTDCLNKGLSIQGVTITIPYDKQVPGLPAQPGAGGGHMSPSNVAQVLSHYKGALKGLMTWSLNWDGSKNWTFGDNVKGTLGTA

SEQ ID NO: 61 - DNA, 75 bp, Artificial sequence
  Plant secretion leader peptide
atggagagctttcgacggttctctctcctgagttttattgcgctgcttgcctactttgccttcttggcttctgct

SEQ ID NO: 62 - DNA, 75 bp, Artificial sequence
  Plant secretion leader peptide
atggagtctttcagacggttttcgctccttagcttcatcgctcttctggcctatttcgctttcttggcttccgca

SEQ ID NO: 63 - DNA, 75 bp, Artificial sequence
  Plant secretion leader peptide
atggagagctttcgacggttttccctcttgtcctttattgccctgttggcgtattttgccttcttggcttctgct

SEQ ID NO: 64 - DNA, 75 bp, Artificial sequence
  Plant secretion leader peptide
atggagagctttcgacgcttttccctcttgagcttcatcgccttgctcgcgtatttcgctttcttggcctccgct

SEQ ID NO: 65 - DNA, 75 bp, Artificial sequence
  Plant secretion leader peptide
atggagtctttcagacgcttctccctcttgagttttattgcgctgcttgcctatttcgcctttcttgcttctgct

SEQ ID NO: 66 - DNA, 75 bp, Artificial sequence
  Plant secretion leader peptide
atggagagctttcgacggttctccctcttgtcttttatagccctgctcgcctatttcgctttcttggcttcggct

SEQ ID NO: 67 - DNA, 75 bp, Artificial sequence
  Plant secretion leader peptide coding sequence
atgggccttcaacagggcttggttacttggtttgttggcgtcctgtttctctctactctccttttgtcgaacgca

SEQ ID NO: 68 - DNA, 75 bp, Artificial sequence
  Plant secretion leader peptide coding sequence
atgggactgcagcaaggcttggttacatggtttgttggcgtcctgtttctctctactctccttttgagcaacgcc

(19) United States
(12) Patent Application Publication
(10) Pub. No.: US 2013/0097731 A1
(43) Pub. Date: Apr. 18, 2013

(54) PEST-RESISTANT PLANTS CONTAINING A COMBINATION OF A SPIDER TOXIN AND A CHITINASE

(71) Applicants: Dror Avisar, Kochav Yair (IL); Hanan Stein, Nes-Ziona (IL); Ziv Shani, Mazkeret Batia (IL)

(72) Inventors: Dror Avisar, Kochav Yair (IL); Hanan Stein, Nes-Ziona (IL); Ziv Shani, Mazkeret Batia (IL)

(73) Assignee: Futuragene Israel Ltd., Rechovot (IL)

(21) Appl. No.: 13/704,729

(22) PCT Filed: Jun. 16, 2011
(86) PCT No.: PCT/IL2011/000483

§ 371 Date: Dec. 17, 2012

Related U.S. Application Data
(60) Provisional application No. 61/355,208, filed on Jun. 16, 2010.

(51) Int. Cl. C12N 15/82

(52) U.S. Cl. CPC C12N 15/8286
USPC 800/279; 536/23.2; 435/320.1; 800/302; 424/94.61

(57) ABSTRACT

An isolated polynucleotide is disclosed comprising:
(i) a nucleic acid sequence encoding at least one toxic peptide, the toxic peptide being a spider toxin; and
(ii) a nucleic acid sequence encoding a chitinase attached to a secretion signal sequence.

Uses thereof and plants expressing same are also disclosed.

### FIELD AND BACKGROUND OF THE INVENTION

The present invention, in some embodiments thereof, relates to pest-resistant plants and methods of generating same.

Pests are responsible for massive yield lost by foraging on plant tissues and by transmitting microbe and viral diseases. Insecticidal agents and compositions have been developed to control insect pests such as agrohorticultural pests, hygienic pests, or wood-eating pests and in practice have been used as a single or a mixed agent.

However, it is believed that about one billion dollars are lost annually in the field due to pest attack, even with chemical control. Since the introduction of DDT in the 1940s, insect pests have been controlled almost exclusively by a limited number of broad-spectrum chemical insecticides that have led to the development of resistant populations in several important pest groups. The abusive and indiscriminate use of chemical insecticides is responsible for environmental and ecological damage to natural enemies and the death of millions of birds and fishes along with the insect-pests. In addition, there is strong epidemiological and experimental evidence linking the occurrence of cancer, Parkinson's disease and others neurological disorders to pesticide exposure. Thus, it is important at this juncture to identify and characterize novel compounds with insecticidal activity.

Spider venom contains a vast array of biologically active substances, some of which are toxins. They are thought to be a rich source of insecticidal compounds since the primary action of spider venom is to kill or paralyze arthropod prey by targeting the nervous system of these organisms. The specificity of some spider toxins acting only in insects has an enormous potential for application as bioinsecticides. The x-ACTX-Hv1a toxin (Hvt) found in the venom of the Australian funnel web spider (Hadronyche versuta) is an insect-specific calcium-channel antagonist (Norton R S, Toxicon, 1998, 36, 1573-83). The peptide is toxic to a range of agriculturally important arthropods in the Coleoptera, Lepidoptera and Diptera orders and has been reported to have no effects on a number of mammals (Norton R S, Toxicon, 1998, 36, 1573-83). Recently, active recombinant spider toxins have been cloned and expressed in prokaryotic (Fitches E. et al., Insect. Biochem. Mol. Biol., 2002, 32, 1653-61, Khan et al., Transgenic Res., 2006, 15, 349-57) and eukaryotic systems (Fitches E. et al., Insect. Biochem. Mol. Biol., 2002, 32, 1653-61), and transgenic plants expressing spider insecticidal peptides are resistant to insect attack (Khan et al., Transgenic Res., 2006, 15, 349-57).

The use of transgenic plants to control pests reduces or eliminates the need to use externally applied chemical pesticides which is often not practical or economically feasible for certain species such as large forestry tree species. Furthermore, the use of transgenic plants can effectively target pests that are otherwise not readily accessible to externally applied pesticides. For example, certain pests reside in protective galls and/or penetrate into the plant tissues via tunneling or other elusive mechanisms which partially or fully shield the pest from the externally applied toxins. Insect resistant-transgenic crops, including rice, expressing Bacillus thuringiensis (Bt) Cry toxins have been successfully and widely used for more than a decade. These toxins are highly active against coleopteran, dipteran and lepidopteron insect but lack any effective activity against hymenoptera pests (wasps and ants).

Spider venom consists of a complex mixture of substances containing a variety of toxic components. Polypeptides with insecticidal activity isolated from the venom glands of different spider species display spatial structure homology and interact with ion channels of the excitable membrane, affecting its functioning.

The transgenic expression of ω-ACTX-Hv1a toxin (Hvt) in tobacco effectively protected the plants from Helicoverpa armigera and Spodoptera littoralis larvae, with 100% mortality within 48 hours (Khan S. A. et al., Transgenic Res., 2006, 15, 349-57).

The usage of arthropod neurotoxins in transgenic plants to control tissue-chewing pests has until presently not been very successful.

Insects are protected by a hardened outer skeletal surface made of chitin. Chitin, together with additional proteins is also found in the peritrophic membrane (PM), a film-like structure that separates food from midgut tissue. It protects the epithelium against food abrasion and microrganisms and has other functions based on compartmentalization of enzymes. Chitinases are enzymes that degrade chitin but by themselves are not capable of controlling tissue chewing pests (Shakhbazau, (BLR), Rus. J. Genet. 2008, 44: 1013-22). Developmental effects on insects that were fed chitinase-transgenic plants were mild to severe, depending on the stiffness of the digested tissue that physically harmed the gut, but the plants were not totally resistant to pests.

U.S. Pat. No. 7,196,057 teaches plants expressing fusion proteins comprising a translocating moiety such as lectin and a moiety which is toxic to insects, including chitinase.

U.S. Patent Application No. 20020197689 teaches insecticidal peptides including peptides derived from Pireneitega luctuosa.

U.S. Pat. No. 5,177,308 teaches plants expressing insecticidal peptides including peptides derived from Agelenopsis aperta.

WO9949035A2 teaches plants expressing insecticidal peptides including peptides derived from Segestria florentina.

### SUMMARY OF THE INVENTION

According to an aspect of some embodiments of the present invention there is provided an isolated polynucleotide comprising:

(i) a nucleic acid sequence encoding at least one toxic peptide, the toxic peptide being a spider toxin; and

(ii) a nucleic acid sequence encoding a chitinase attached to a secretion signal sequence.

According to an aspect of some embodiments of the present invention there is provided an isolated polynucleotide comprising:

(i) a nucleic acid sequence encoding at least one toxic peptide, the toxic peptide not being chitinase; and

(ii) a nucleic acid sequence encoding a chitinase.

According to an aspect of some embodiments of the present invention there is provided a nucleic acid construct comprising an isolated polynucleotide of the present invention and a cis regulatory element.

According to an aspect of some embodiments of the present invention there is provided a nucleic acid construct system comprising:

(i) a first nucleic acid construct comprising an isolated polynucleotide which comprises a nucleic acid sequence encoding a toxic peptide and a cis regulatory element; and

(ii) a second nucleic acid construct comprising an isolated polynucleotide which comprises a nucleic acid sequence encoding a chitinase and a cis regulatory element.

According to an aspect of some embodiments of the present invention there is provided an isolated polypeptide comprising a toxic peptide which comprises a sequence at least 90% homologous, and/or at least 80% identical to a sequence selected from the group consisting of SEQ ID NOs: 9, 15, 24, 30, 55 and 56-60 as determined using the BlastP software of the National Center of Biotechnology Information (NCBI) using default parameters, the toxic peptide being attached to a plant lectin.

According to an aspect of some embodiments of the present invention there is provided an insecticidal composition comprising:
(i) a toxic peptide; and
(ii) a chitinase

According to an aspect of some embodiments of the present invention there is provided a method of controlling or exterminating an insect, the method comprising expressing in a host plant of the insect any of the isolated polynucleotides of the present invention, thereby controlling or exterminating the insect.

According to an aspect of some embodiments of the present invention there is provided a method of controlling or exterminating an insect, the method comprising expressing in a host plant of the insect an isolated polynucleotide which comprises a nucleic acid sequence encoding a toxic peptide selected from the group consisting of SEQ ID NOs: 9, 15, 24, 30 and 55-57.

According to an aspect of some embodiments of the present invention there is provided a method of controlling or exterminating an insect, the method comprising contacting the insect with any of the insecticidal compositions of the present invention, thereby controlling or exterminating the insect.

According to some embodiments of the invention, the toxic peptide is derived from insects selected from the group consisting of bees, wasps, cockroach, blowfly, mosquito, webworm, beetle, antipode, millipede, crab, lobster, shrimp, prawn, spider, scorpion, mite and tick.

According to some embodiments of the invention, the toxic peptide is derived from a spider.

According to some embodiments of the invention, the toxic peptide comprises an amino acid sequence at least 90% homologous, and/or at least 80% identical to a sequence selected from the group consisting of SEQ ID NOs: 9, 15, 24, 30, 55, 56 and 57 as determined using the BlastP software of the National Center of Biotechnology Information (NCBI) using default parameters.

According to some embodiments of the invention, the toxic peptide is attached to a plant lectin.

According to some embodiments of the invention, the chitinase is not attached to a plant lectin.

According to some embodiments of the invention, the plant lectin comprises Galanthus nivalis agglutinin (GNA).

According to some embodiments of the invention, the toxic peptide is attached to a secretion signal sequence.

According to some embodiments of the invention, the chitinase is attached to a secretion signal sequence.

According to some embodiments of the invention, the secretion signal sequence is encoded by a nucleic acid as set forth in SEQ ID NOs: 7, 21 and 61-68.

According to some embodiments of the invention, the chitinase comprises an amino acid sequence at least 90% homologous, and/or at least 80% identical to a sequence selected from the group consisting of SEQ ID NO: 36, 42 and 58-60, as determined using the BlastP software of the National Center of Biotechnology Information (NCBI) using default parameters.

According to some embodiments of the invention, the at least one toxic peptide comprises a first and a second toxic peptide, wherein the first toxic peptide targets a first site in a sodium (Naᵥ) channel and the second toxic peptide targets a second site in the Naᵥ channel.

According to some embodiments of the invention, the at least one toxic peptide comprises a first, second and a third toxic peptide, wherein the first toxic peptide targets a first site in a sodium (Naᵥ) channel, the second toxic peptide targets a second site in the Naᵥ channel and the third toxic peptide targets a third site in the Naᵥ channel.

According to some embodiments of the invention, the first toxic peptide is P83591, the second toxic peptide is P83558 and the third toxic peptide is P11060.

According to some embodiments of the invention, the at least one toxic peptide comprises at least 4 toxic peptides, wherein a first of the at least 4 toxic peptides is P83591, a second of the at least 4 toxic peptides is P83558, a third of the at least 4 toxic peptides is P11060, a first of the at least 4 toxic peptides is P61095.

According to some embodiments of the invention, the toxic peptide is attached to a plant lectin.

According to some embodiments of the invention, the toxic peptide is attached to a secretion signal sequence.

According to some embodiments of the invention, the chitinase is attached to a secretion signal sequence.

According to some embodiments of the invention, the isolated polypeptide further comprises a secretion signal peptide.

According to some embodiments of the invention, the isolated polynucleotide comprises a nucleic acid sequence encoding an isolated polypeptide of the invention.

According to some embodiments of the invention, the isolated polynucleotide further comprises a nucleic acid sequence encoding a chitinase.

According to some embodiments of the invention, the nucleic acid construct comprises the isolated polynucleotide of the present invention and a cis regulatory element.

According to some embodiments of the invention, the nucleic acid construct system comprises:

(i) a nucleic acid construct of the present invention; and

(ii) a nucleic acid construct comprising an isolated polynucleotide which comprises a nucleic acid sequence encoding a chitinase.

According to some embodiments of the invention, the cis-regulatory element is a promoter.

According to some embodiments of the invention, the promoter is SVBV or sgFiMV.

According to some embodiments of the invention, the promoter is a plant promoter.

According to some embodiments of the invention, the plant promoter is a leaf-specific promoter.

According to some embodiments of the invention, the plant comprises the nucleic acid construct of the present invention.

According to some embodiments of the invention, the plant comprises the nucleic acid construct system of the present invention.

According to some embodiments of the invention, the plant is a tree.

According to some embodiments of the invention, the plant is a eucalyptus tree.

According to some embodiments of the invention, the insecticidal composition comprises the isolated polypeptide of the present invention.

According to some embodiments of the invention, the insect comprises a sessile gall nesting insect.

According to some embodiments of the invention, the sessile gall nesting insect comprises a gall wasp.

According to some embodiments of the invention, the expressing is effected using a nucleic acid construct comprising a leaf-specific promoter.

According to some embodiments of the invention, the host plant comprises a tree.

According to some embodiments of the invention, the tree is a eucalyptus tree.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are schemes illustrating the construction of an expression vector according to one embodiment of the present invention (vector #257). FIG. 1A illustrates the synthetic fragment and FIG. 1B illustrates the vector in which it is inserted.

FIG. 2 is a photograph of a restriction analysis confirming the insertion and correct orientation of insert AntiInsects6Cassete into the vector.

FIGS. 3A-C are schemes illustrating the construction of an expression vector according to one embodiment of the present invention (vector #258). FIGS. 3A and 3B illustrates the synthetic fragments and FIG. 3C illustrates the vector in which it is inserted.

FIG. 4 is a photograph of a restriction analysis confirming the insertion of insert AntiInsects6CDS GNA into the vector.

FIG. 5 is a scheme illustrating two exemplary constructs of the present invention, with annotated sequences.

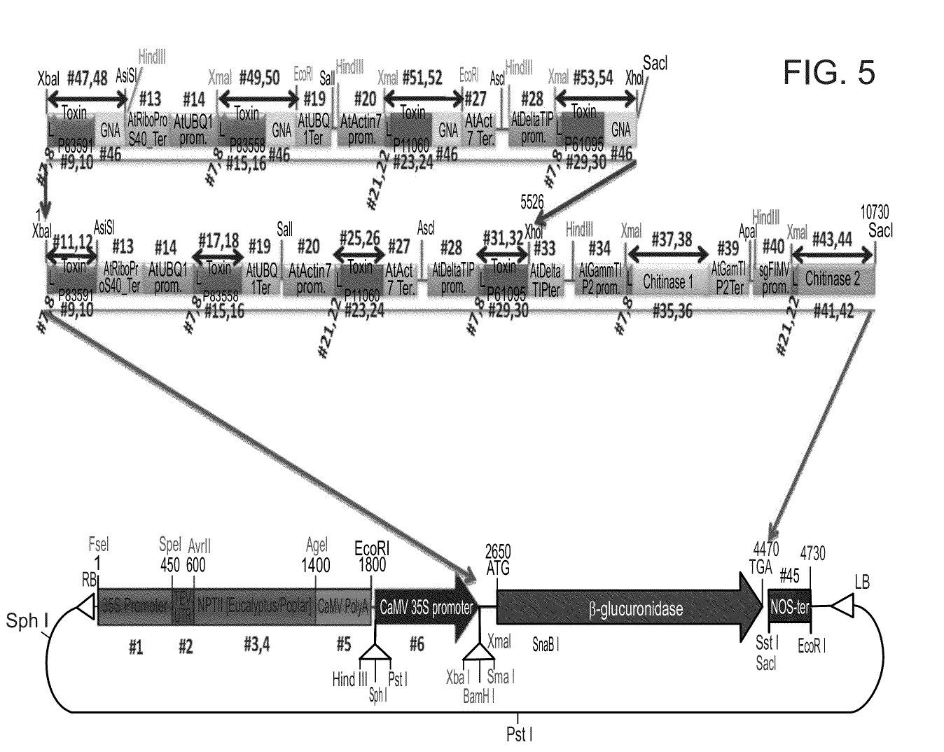

### DESCRIPTION OF SPECIFIC EMBODIMENTS OF THE INVENTION

The present invention, in some embodiments thereof, relates to pest-resistant plants and methods of generating same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Insect-pests are global problems that cause severe damage to crop plants, and their control is commonly based on chemical insecticides. However, negative effects of pesticides on the environment and human health emphasize the necessity to develop alternative methods for insect-pest control.

Pests are responsible for massive yield lost by foraging on plant tissues and by transmitting microbe and viral diseases.

The use of transgenic plants to control pests reduces or eliminates the need to use externally applied chemical pesticides which is often not practical or economically feasible for certain species such as large forestry tree species. Furthermore, the use of transgenic plants can effectively target pests that are otherwise not readily accessible to externally applied pesticides. For example, certain pests reside in protective galls and/or penetrate into the plant tissues via tunneling or other elusive mechanisms which partially or fully shield the pest from the externally applied toxins.

One particular toxin which has been expressed in plants for the purpose of controlling pests is chitinase. This toxin acts by both dissolving both the outer cuticle of the insect pest and its peritrophic membrane (PM), the film-like structure that separates food from midgut tissue.

However, soft tissue and sap feeding insects are not likely to be affected by the disruption of the PM. Accordingly, the present inventors propose the co-expression of chitinases and spider neurotoxins in plants to control herbivorous pests. The chitinases would interfere with the intact chitin, improving the chance of the spider toxin to penetrate the hemolymph.

The present inventors further propose fusion of the spider neurotoxin to a plant secretion leader peptide. This would allow the toxin to be translated in the ER pathway and to be secreted to the extracellular matrix. In this way, sap feeding and gall nesting pests which may be protected from inner cellular agents might be exposed to the spider neurotoxins not only by digestion of plant material but also by its outer surface. Thus, gall nesting pests developing inside the galls would be exposed to the toxin for a long time through both digestion and cuticle absorption.

Thus, according to a first aspect of the invention there is provided a method of controlling or exterminating an insect, the method comprising expressing in a host plant of the insect a chitinase and at least one toxic peptide, thereby controlling or exterminating the insect.

Contemplated insects for control or extermination include those that affect the growth, development, reproduction, harvest, yield or utility of a plant.

According to one embodiment the insects to be eradicated are sessile insects. According to another embodiment, the insects are gall nesting insects, such as for example sessile gall wasps (Cynipidae). Particular contemplated species of sessile gall wasps include, but are not limited to Leptocybe invasa, Ophelimus maskelli and Selitrichodes globulus. Other insects for control or extermination include, but are not limited to Coleopterans eg. Southern corn rootworm (Diabrotica undecimpunctata); cowpea bruchid (Callosobruchus maculatus); Lepidopterans eg. European cornborer (Ostinia nubilalis); tobacco hornworm (Manduca sexta); stem borer (Chilo partellus):

Homopteran pests eg. Rice brown plant hopper (Nilaparvata lugens); rice green leaf hopper (Nephotettix cinciteps); potato leaf hopper (Empoasca fabae); peach potato aphid (Myzus persicae).

The term “plant” as used herein encompasses whole plants, ancestors and progeny of the plants and plant parts, including seeds, shoots, stems, roots (including tubers), and plant cells, tissues and organs. The plant may be in any form including suspension cultures, embryos, meristematic regions, callus tissue, leaves, gametophytes, sporophytes, pollen, and microspores. Plants that are particularly useful in the methods of the invention include all plants which belong to the superfamily Viridiplantee, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalyptus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively algae and other non-Viridiplantae can be used for the methods of the present invention. According to a specific embodiment, the tree is a eucalyptus tree.

As mentioned, the method of the present invention is affected by co-expressing at least one toxic peptide and at least one chitinase in the plant.

In order to express the toxic peptides and chitinases in plants, polynucleotides encoding same are inserted into expression constructs and introduced into the plants (plant transformation) such that expression occurs in the plant, as further described herein below.

The present invention contemplates expressing one, two, three, four or more toxic peptides. Toxic peptides include any peptide (including its metabolic precursor or pro-agent), that affects the wellbeing, growth or reproduction of an insect and/or any stages of its life cycle. According to one embodiment, the toxic peptide is derived from insects or related arthropods.

In a preferred embodiment of the invention the toxic peptide that is expressed in the plant is in its mature form and, ideally, is a natural or synthetic arthropod-derived peptide or protein or metabolite or analogue thereof, capable of causing deleterious effects on growth, development reproduction or mortality in pest insects; such as an insect or related arthropod or the like derived protein or peptide or neuropeptide or metabolite or analogue thereof.

Suitably the toxic peptide is derived from insects such as cockroach, blowfly, mosquito, webworm, beetle, wasps, bees, or related arthropods such as antipode, millipede, crab, lobster, shrimp, prawn, spider, scorpion, mite, tick and the like.

Exemplary insect toxins that may be used to carry out the present invention are described in U.S. Pat. No. 6,162,430 and U.S. Pat. No. 7,196,057, the contents of both being incorporated herein by reference.

According to one embodiment, the toxic peptide is selectively toxic towards a particular insect. Such toxins are further described by Nicholson (Toxicon 49 (2007) 490-512), the contents of which are incorporated herein by reference.

According to another embodiment, the toxic peptide is selectively toxic towards non-vertebrates.

Suitable toxic insect peptides include any one or more of the following neuropeptides and their natural or synthetic metabolites or analogues: Manduca sexta allatostatin (Manse-AS); cockroach allatostatin such as those found in either of the following species Diplotera punctata or Periplaneta americana or blowfly allatostatin such as in the species Calliphora vomitaria; alternatively, peptides comprising, or derived from, insect diuretic hormones such as those isolated from any one or more of the aforementioned species, or related arthropod hormones may be used.

A useful scorpion toxin is, for example, AaIT from Androctonus australis. Zlotkin et al., Biochimie, 53, 1073-1078 (1971). An useful snail venom is that from the snail Conus querciones, which the animal delivers by mouth and some individual toxins of which appear to be selective for arthropods including insects. See, for example, Olivera et al., “Diversity of Conus Neuropeptides,” Science, 249:257-263 (1990).

According to a particular embodiment, the toxic peptide is a spider toxin (e.g a spider neurotoxin).

The spider neurotoxin may act by targeting a voltage gated sodium (Nay) channel of the insect.

Exemplary insect selective spider toxin include, but are not limited to hainantoxin (which targets site 1 of the Naᵥ channel); Tx4(6-1) and Magi 2 (which target site 3 of the the Naᵥ channel); and δPalutoxin (which targets site 4 of the the Naᵥ channel).

As mentioned, the present invention contemplates expressing one, two, three, four or more toxic peptides. According to a specific embodiment, each peptide which is expressed targets a different site, thus allowing synergistic effects of the toxic peptides. Thus, for example, the present invention contemplates selecting spider toxins which target different sites on the voltage gated sodium channel such as P83591 (targets site 1 of the sodium channel), P83558 (targets site 3 of the sodium channel) and P11060 (targets site 4 of the sodium channel).

An exemplary combination of spider toxin that may be expressed in the plant is P83591, P83558, P11060 and P61095.

According to a preferred embodiment, the insect peptide is derived from Haplopelma hainanum, Macrothele gigas, Phoneutria nigriventer, Pireneitega luctuosa, Agelenopsis aperta or Segestria florentina.

Contemplated peptide toxins include those having a sequence at least 90% homologous, and/or at least 80% identical to a sequence selected from the group consisting of SEQ ID NOs: 9, 15, 24, 30, 55, 56 and 57 as determined using the BlastP software of the National Center of Biotechnology Information (NCBI) using default parameters.

The choice of toxic peptide will be determined by the nature of the pathogen to be destroyed. For example, the size of a toxic agent may be chosen on the basis of the type of gut wall to be penetrated and the effectiveness of the toxic agent will be based on the type of insect to be destroyed.

According to one embodiment the toxic peptides of this aspect of the present invention are attached to a plant lectin.

According to a particular embodiment, the toxic peptide and/or the chitinase is upstream to the lectin.

Suitable plant lectins which may be attached to the toxic peptides and/or chitinases include any that are capable of penetrating into the insect gut. The choice of which type of lectin to be selected depends on its stability in the insect gut, the type of gut wall to be penetrated and its level of toxicity; a non toxic lectin is preferred.

Examples of plant lectins include, but are not limited to snowdrop lectin (GNA), pea lectin Pisum sativum (P-lec), peanut lectin Arachis hypogaea, french bean lectin (PHA, phytohaemo glutinin), Zephyranthes candida lectin, Amaryllis minuta lectin, Hippeastrum vittatum lectin, Clivia miniata lectin, Lycoris radiate lectin, Narcissus tazetta lectin and Narcissus hybrid lectin and analogues thereof.

In a preferred embodiment of the invention the plant protein is selected from the following group of proteins: GNA (snowdrop lectin; SEQ ID NO: 46); P-lec pea lectin; and peanut lectin.

Preferably the toxic peptides and lectin are linked together by genetic or biochemical means and so, in the first instance, by at least one linking peptide or, in the second instance, by a covalent or non-covalent bond or linking moiety. Where a peptide is used to link the members together the number of peptides is determined by the distance between the relevant ends of each member when the fusion protein is in a biologically active conformation. The moieties may be releasably linked by means adapted to dissociate and release the toxic agent in situ in an insect gut, for example on metabolisation by the insect or may remain intact, depending on the active form of the toxic agent.

Thus an exemplary polynucleotide of the present invention is one set forth in SEQ ID NO: 47 which encodes a Fused Plant secretion leader peptide from sp|Q56YT0|LAC3_At Laccase+Spider toxin P83591+GNA, as set forth in SEQ ID NO: 48. Another exemplary polynucleotide of the present invention is one set forth in SEQ ID NO: 49 which encodes a Fused Plant secretion leader peptide from sp|Q56YT0|LAC3_At Laccase+Spider toxin P83558+GNA, as set forth in SEQ ID NO: 50. Another exemplary polynucleotide of the present invention is one set forth in SEQ ID NO: 51 which encodes a Fused Plant secretion leader peptide from tr|Q6TDS6|Q6TDS6_GOSAR Secretory laccase Gossypium arboreum+Spider toxin P11060+GNA, as set forth in SEQ ID NO: 52. Another exemplary polynucleotide of the present invention is one set forth in SEQ ID NO: 53 which encodes a Fused Plant secretion leader peptide from sp|Q56YT0|LAC3_At Laccase+Spider toxin P61095+GNA, as set forth in SEQ ID NO: 54.

As mentioned, the method of the present invention is affected by co-expressing a chitinase and a toxic peptide in a plant.

As used herein, the term “chitinase” refers to an enzyme which digests chitin [poly(β-1,4-N-acetyl D-glucosamine)] to generate oligosaccharides and N-acetylglucosamine. In order to test whether a polypeptide comprises chitinase activity, the polypeptide may be brought into contact with a substrate of chitinase, and then the digestion and/or a degree thereof of the chitinase substrate is analyzed [for example, Johannes et al., Infect. Immun., 69, 4041-4047 (2001)]. For example, a polypeptide to be tested is added to a well of an agarose gel containing an appropriate substrate of chitinase (for example, glycol chitin or chitin), and incubated for a predetermined period (for example, at 37° C. for 12 hours). The gel is stained with an appropriate dye [for example, Fluorescent Brightener 28 (Sigma)] and observed under an ultraviolet ray. The portion in which chitin is digested by chitinase does not react with the dye, and becomes black. In this case, it may be judged that the polypeptide to be tested exhibits the chitinase activity. Conversely, when the chitinase reaction does not occur, the gel is brightened by the reaction with the dye. In this case, it may be judged that the polypeptide to be tested does not exhibit the chitinase activity.

Suitable chitinases that may be co-expressed in the plants include insect chitinase such as those for example, found in M. sexta; Bombyx mori; the mosquito Anopheles gambiae; fall webworm Hyphantria cunea; beetle Phaedon cochleariae; or Lacanobia oleracea.

According to one embodiment, the chitinase is derived from an organism which digests insect chitin as part of its diet. Thus, for example chitinases from the plant Nepenthes khasiana and the fungus Beauveria bassiana are contemplated for use in the present invention.

Chitinases have been isolated from many plant species and they are classified into 5 classes (I-V) according to their multi-domain structure (Collinge et al., 1993; Hamel et al., 1997) and the present invention contemplates the use of any of these classes.

Class I chitinases are mainly composed of basic proteins (with basic pI values), mostly targeted to the vacuoles and found in both monocots and dicots. These enzymes display high specific activities and are responsible for the majority of the plant chitinolytic activity in roots, shoots and flowers (Legrand et al., 1987). Class I chitinases are composed of five structural domains: (i) N-terminal signal peptide (20-27 amino acids residues) that routes the protein into the endoplasmic reticulum; (ii) cysteine rich domain (CRD of about 40 amino acids), which is involved in chitin binding and contains eight cysteine residues in highly conserved positions; (iii) proline (mostly hydroxyproline)-rich hinge region (HR) that varies in size; (iv) catalytic domain (CD>220 amino acids), comprising the central domain of the protein that shows high homology to the catalytic domain of class II and IV chitinases and low homology to the CD of bacterial chitinases; and (v) carboxy-terminal extension (CTE), which targets the protein into the vacuole and is present in most of class I chitinases (Graham and Sticklen, 1994; Hamel et al., 1987). Rapid release of large amounts of the vacuole-compartmentalized chitinase occurs during cell lysis resulting from hypersensitive response to pathogen invasion. Several class I-basic chitinases, which are devoid of CTE, have also been characterized. These chitinases are secreted to the extracellular space (Legrand et al., 1987; Swegle et al., 1992; Vad et al., 1991).

Class II chitinases are acidic (with acidic pI), containing only the signal peptide and catalytic domain. The latter shows a high amino acid sequence homology to the catalytic region of class I and class IV chitinases. The specific activity of acidic chitinases is lower than that of class I-chitinases. It is assumed that the primary function of class II-chitinases is to generate elicitors of defense responses by partial degradation of the fungal pathogen cell wall (Graham and Sticklen, 1994).

Class III chitinases include basic or acidic extracellular proteins with chitinase/lysozyme activity. Their catalytic domain is different from that of class I and II but shares significant identity with chitinases from yeast and filamentous fungi.

Class IV chitinases share structural domain similarity with class I chitinases but not a high amino acid sequence identity. All of class IV enzymes lack the CTE and are therefore targeted to the apoplast. In addition, amino acid sequence alignment with class I proteins showed four distinct deletions; one in the chitin binding domain and three within the catalytic domain. This group include the PR4 chitinase from bean, the ChB4 from Canola and many others (Hamel et al., 1997).

Class V chitinases share some homology to exo-chitinases of bacterial origins, e.g. Serracia marcescens, Bacillus circulans and Streptomyces plicatus.

Exemplary chitinase sequences include but are not limited to those having a sequence at least 90% homologous, and/or at least 80% identical to a sequence selected from the group consisting of SEQ ID NOs: 36, 42 and 58-60 as determined using the BlastP software of the National Center of Biotechnology Information (NCBI) using default parameters.

The present invention contemplates attaching either the toxic peptide, the chitinase or both to a signal peptide. According to one embodiment, the signal peptide is a secretion signal peptide such that they are excreted into the extracellular matrix.

As used herein, the phrase “signal peptide” refers to a peptide linked in frame to the amino terminus of a polypeptide and directs the encoded polypeptide into a cell's secretory pathway.

Thus, the present invention contemplates the polynucleotide sequence SEQ ID NO: 11 which encodes for SEQ ID NO: 12 (fused plant secretion leader peptide form sp. Q56YTO/LAC3_At Laccase and spider toxin P83591); the polynucleotide sequence SEQ ID NO: 17 which encodes for SEQ ID NO: 18 (fused plant secretion leader peptide form sp. Q56YTO/LAC3_At Laccase and spider toxin P83558); the polynucleotide sequence SEQ ID NO: 25 which encodes for SEQ ID NO: 26 (fused plant secretion leader peptide from tr|Q6TDS6|Q6TDS6_GOSAR Secretory laccase Gossypium arboreum and spider toxin P11060); the polynucleotide sequence SEQ ID NO: 31 which encodes for SEQ ID NO: 32 (fused plant secretion leader peptide form sp. Q56YTO/LAC3_At Laccase and spider toxin P61095); the polynucleotide sequence SEQ ID NO: 37 which encodes for SEQ ID NO: 38 (fused plant secretion leader peptide form sp. Q56YTO/LAC3_At Laccase and Beauveria bassiana chitinase gb/ACF32998.1; the polynucleotide sequence SEQ ID NO: 43 which encodes for SEQ ID NO: 44 (fused plant secretion leader peptide from tr|Q6TDS6|Q6_TDS6_GOSAR Secretory laccase Gossypium arboreum and Beauveria bassiana endochitinase gb/AAN41261.1.

As mentioned, in order to express the toxic peptides and chitinases in plants, polynucleotides encoding same are introduced into the plants (plant transformation) such that expression occurs in the plant.

Nucleic acid sequences according to this aspect of the present invention can be a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

The nucleic acid sequences encoding the toxic peptide and chitinase according to this aspect of the present invention may be altered, to further improve expression levels in plant expression system. For example, the nucleic acid sequence of the toxic peptide and/or chitinase may be modified in accordance with the preferred codon usage for plant expression. Increased expression of the toxic peptide and/or chitinase in plants may be obtained by utilizing a modified or derivative nucleotide sequence. Examples of such sequence modifications include, but are not limited to, an altered G/C content to more closely approach that typically found in plants, and the removal of codons atypically found in plants commonly referred to as codon optimization.

The phrase “codon optimization” refers to the selection of appropriate DNA nucleotides for use within a structural gene or fragment thereof that approaches codon usage within a plant. Therefore, an optimized gene or nucleic acid sequence refers to a gene in which the nucleotide sequence of a native or naturally occurring gene has been modified in order to utilize statistically-preferred or statistically-favored codons within a plant. The nucleotide sequence typically is examined at the DNA level and the coding region optimized for expression in plants determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native heparanase gene relative to that of highly expressed plant genes, followed by a calculation of the average squared deviation. The formula used is: 1 SDCU=n=1 N[(Xn−Yn)/Yn]2/N, where Xn refers to the frequency of usage of codon n in highly expressed plant genes, where Yn to the frequency of usage of codon n in the gene of interest and N refers to the total number of codons in the gene of interest. A table of codon usage from highly expressed genes of dicotyledonous plants is compiled using the data of Murray et al. (1989, Nuc Acids Res. 17:477-498).

The nucleic acid sequence encoding the toxic peptide and/or chitinase may be altered, to further improve expression levels for example, by optimizing the nucleic acid sequence in accordance with the preferred codon usage for a particular plant cell type which is selected for the expression of the toxic peptide and/or chitinase polypeptide. Use of tobacco plants for the expression of the toxic peptide and/or chitinase (as described in the Examples section hereinbelow) may limit the need for optimizing the nucleic acid sequence in accordance with the preferred codon usage since tobacco plant codon usage/preference is generally very similar to humans

One method of optimizing the nucleic acid sequence in accordance with the preferred codon usage for a particular plant cell type is based on the direct use, without performing any extra statistical calculations, of codon optimization tables such as those provided on-line at the Codon Usage Database through the NIAS (National Institute of Agrobiological Sciences) DNA bank in Japan (www.kazusa.or.jp/codon/). The Codon Usage Database contains codon usage tables for a number of different species, with each codon usage table having been statistically determined based on the data present in Genbank

By using the above tables to determine the most preferred or most favored codons for each amino acid in a particular species (for example, rice), a naturally-occurring nucleotide sequence encoding a protein of interest can be codon optimized for that particular plant species. This is effected by replacing codons that may have a low statistical incidence in the particular species genome with corresponding codons, in regard to an amino acid, that are statistically more favored. However, one or more less-favored codons may be selected to delete existing restriction sites, to create new ones at potentially useful junctions (5′ and 3′ ends to add signal peptide or termination cassettes, internal sites that might be used to cut and splice segments together to produce a correct full-length sequence), or to eliminate nucleotide sequences that may negatively effect mRNA stability or expression.

The naturally-occurring or native toxic peptide and/or chitinase encoding nucleotide sequence may already, in advance of any modification, contain a number of codons that correspond to a statistically-favored codon in a particular plant species. Therefore, codon optimization of the native toxic peptide and/or chitinase nucleotide sequence may comprise determining which codons, within the native the toxic peptide and/or chitinase nucleotide sequence, are not statistically-favored with regards to a particular plant, and modifying these codons in accordance with a codon usage table of the particular plant to produce a codon optimized derivative. The modified or derivative nucleotide sequence encoding the toxic peptide and/or chitinase may be comprised, 100 percent, of plant preferred codon sequences, while encoding a polypeptide with the same amino acid sequence as that produced by the native toxic peptide and/or chitinase coding sequence. Alternatively, the modified nucleotide sequence encoding the toxic peptide and/or chitinase may only be partially comprised of plant preferred codon sequences with remaining codons retaining nucleotide sequences derived from the native toxic peptide and/or chitinase coding sequence. A modified nucleotide sequence may be fully or partially optimized for plant codon usage provided that the protein encoded by the modified nucleotide sequence is produced at a level higher than the protein encoded by the corresponding naturally occurring or native gene. For example, the modified toxic peptide and/or chitinase may comprise from about 60% to about 100% codons optimized for plant expression. As another example, the modified toxic peptide and/or chitinase may comprise from 90% to 100% of codons optimized for plant expression.

Construction of synthetic genes by altering the codon usage is described in for example PCT Patent Application 93/07278.

Constructs (or vectors) useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. The genetic construct can be an expression vector wherein the heterologous nucleic acid sequence is operably linked to a cis-acting regulatory element allowing expression in the plant cells.

As used herein, the phrase “cis acting regulatory element” refers to a polynucleotide sequence, preferably a promoter, which binds a trans acting regulator and regulates the transcription of a coding sequence located downstream thereto.

As used herein, the phrase “operably linked” refers to a functional positioning of the cis-regulatory element (e.g., promoter) so as to allow regulating expression of the selected nucleic acid sequence. For example, a promoter sequence may be located upstream of the selected nucleic acid sequence in terms of the direction of transcription and translation.

Preferably, the promoter in the nucleic acid construct of the present invention is a plant promoter which serves for directing expression of the heterologous nucleic acid molecule within plant cells.

As used herein the phrase “plant promoter” refers to a promoter sequence, including any additional regulatory elements added thereto or contained therein, is at least capable of inducing, conferring, activating or enhancing expression in a plant cell, tissue or organ, preferably a monocotyledonous or dicotyledonous plant cell, tissue, or organ. According to one embodiment, the promoter is not a flower promoter (thus protecting bees and other nectar-feeding insects from the toxins).

According to still another embodiment, the promoter is a leaf promoter. Examples of preferred promoters useful for the methods of the present invention include:
sgFiMV—Bhattacharyya (USA), Virus Research, 2002, 90: 47-62.
SVBV—Wang (ISL), Virus Genes, 2000, 20: 11-7; Pattanaik (USA), Plant Science, 2004, 167: 427-38.
PCISV—Maiti (USA), BIOCH. BIOPHYS. RES. COM. 1998, 244: 440-4.
sgMiMV—Dey (USA), Transgenic, 2003, 4: 35-53.
MiMV—Dey (USA), Plant Molecular Biology, 1999, 40: 771-82.
FiMV—Maiti (USA), Transgen. Res. 1997, 6: 143-56.
CsVMV—Verdaguer (USA), AJRTC, 1997, 2: 204-8.
35S promoter from pBI121 (AF485783.1) as set forth in SEQ ID NO: 6.
AtUBQ1 promoter as set forth in SEQ ID NO: 14.
AtActin7 promoter as set forth in SEQ ID NO: 20.
At Delta tip promoter as set forth in SEQ ID NO: 28.
At GammTIP2 promoter as set forth in SEQ ID NO: 34.
Sg FiMV promoter as set forth in SEQ ID NO: 40

Other exemplary promoters are presented in Tables I, II and III.

TABLE I
Exemplary constitutive promoters for use in
the performance of the present invention

| Reference | Expression Pattern | Gene Source |
|---|---|---|
| McElroy etal, Plant Cell, | constitutive | Actin |
| 2: 163-171, 1990 | | |
| Odell et al, Nature, 313: | constitutive | CAMV 35S |
| 810-812, 1985 | | (SEQ ID NO: 1) |
| Nilsson et al., Physiol. | constitutive | CaMV 19S |
| Plant 100: 456-462, 1997 | | |
| de Pater et al, Plant J | constitutive | GOS2 |
| Nov; 2(6): 837-44, 1992 | | |
| Christensen et al, Plant | constitutive | Ubiquitin |
| Mol. Biol. 18: 675-689, | | |
| 1992 | | |
| Bucholz et al, Plant Mol | constitutive | Rice cyclophilin |
| Biol. 25(5): 837-43, 1994 | | |
| Lepetit et al, Mol. Gen. | constitutive | Maize H3 histone |
| Genet. 231: 276-285, | | |
| 1992 | | |
| An et al, Plant J. | constitutive | Actin 2 |
| 10(1); 107-121, 1996 | | |

TABLE II
Exemplary seed-preferred promoters for use
in the performance of the present invention

| | Expression | |
|---|---|---|
| Reference | Pattern | Gene Source |
| Simon, et al., Plant Mol. | seed | Seed specific genes |
| Biol. 5. 191, 1985; | | |
| Scofield, et al., J. Biol. | | |
| Chem. 262: 12202, 1987; | | |
| Baszczynski, et al., Plant | | |
| Mol. Biol. 14: 633, 1990. | | |
| Pearson' et al., Plant | seed | Brazil Nut albumin |
| Mol. Biol. 18: 235-245, | | |
| 1992. | | |
| Ellis, et al. Plant Mol. | seed | legumin |
| Biol. 10: 203-214, 1988 | | |
| Takaiwa, et al., Mol. Gen. | seed | Glutelin (rice) |
| Genet. 208: 15-22, 1986; | | |
| Takaiwa, et al., FEBS | | |
| Letts. 221: 43-47, 1987 | | |
| Matzke et al Plant Mol | seed | Zein |
| Biol, 143). 323-32 1990 | | |
| Stalberg, et al, Planta 199: | seed | napA |
| 515-519, 1996 | | |
| Mol Gen Genet 216: 81- | endosperm | wheat LMW and |
| 90, 1989; NAR 17: 461-2, | | HMW, glutenin-1 |
| Albanietal, Plant Cell, 9: | seed | Wheat SPA |
| 171-184, 1997 | | |
| EMBO3: 1409-15, 1984 | endosperm | wheat a, b and g |
| | | gliadins |
| | endosperm | Barley ltrl promoter |
| Theor Appl Gen 98: 1253- | endosperm | barley B1, C, D |
| 62, 1999; Plant J 4: 343- | | hordein |
| 55, 1993; Mol Gen Genet | | |
| 250: 750-60, 1996 | | |
| Mena et al, The Plant | endosperm | Barley DOF |
| Journal, 116(1): 53-62, | | |
| 1998 | | |
| EP99106056.7 | endosperm | Biz2 |
| Vicente-Carbajosa et al., | endosperm | Synthetic promoter |
| Plant J. 13: 629-640, 1998 | | |
| Wu et al, Plant Cell | endosperm | rice prolamin NRP33 |
| Physiology 39(8) 885- | | |
| 889, 1998 | | |
| Wu et al, Plant Cell | endosperm | rice -globulin Glb-1 |
| Physiology 398) 885-889, | | |
| 1998 | | |
| Sato et al, Proc. Nati. | emryo | rice OSH1 |
| Acad. Sci. USA, 93: | | |
| 8117-8122 | | |
| Nakase et al. Plant Mol. | endosperm | rice alpha-globulin |
| Biol. 33: 513-S22, 1997 | | REB/OHP-1 |
| Trans Res 6: 157-68, 1997 | endosperm | rice ADP-glucose PP |
| Plant J 12: 235-46, 1997 | endosperm | maize ESR gene |
| | | family |
| PMB 32: 1029-35, 1996 | endosperm | sorgum gamma- kafirin |
| Postma-Haarsma ef al, | emryo | KNOX |
| Plant Mol. Biol. 39: 257- | | |
| 71, 1999 | | |
| Wu et at, J. Biochem., | Embryo and | rice oleosin |
| 123: 386, 1998 | aleuton | |
| Cummins, etal., Plant | Seed (embryo | sunflower oleosin |
| Mol. Biol. 19: 873-876, | and dry seed) | |
| 1992 | | |

TABLE III
Alternative rice promoters for use
in the performance of the invention

| expression | gene | PRO # |
|---|---|---|
| transfer layer of embryo + | Metallothionein Mte | PR00001 |
| calli | | |
| transfer layer of embryo | putative beta-amylase | PR00005 |
| Weak in roots | Putative cellulose synthase | PR00009 |
| | lipase (putative) | PR00012 |
| | Transferase (putative) | PR00014 |
| | peptidyl prolyl cis-trans | PR00016 |
| | isomerase (putative) | |
| | unknown | PR00019 |
| | prp protein (putative) | PR00020 |
| | noduline (putative) | PR00029 |
| seed | Proteinase inhibitor Rgpi9 | PR00058 |
| Weak in young flowers | beta expansine EXPB9 | PR00061 |
| young | Structural protein | PR00063 |
| tissues + calli + embryo | | |
| | xylosidase (putative) | PR00069 |
| strong in endosperm | Prolamine 10 Kda | PR00075 |
| strong in endosperm | allergen RA2 | PR00076 |
| strong in endosperm | prolamine RP7 | PR00077 |
| | CBP80 | PR00078 |
| | starch branching enzyme I | PR00079 |
| transfer layer of embryo + | Metallothioneine-like ML2 | PR00080 |
| calli | | |
| shoot | putative caffeoyl-CoA 3-0 | PR00081 |
| | methyltransferase | |
| strong in endosperm | prolamine RM9 | PR00087 |
| strong in endosperm | prolamine RP6 | PR00090 |
| strong in endosperm | prolamine RP5 | PR00091 |
| | allergen RA5 | PR00092 |
| embryo | putative methionine | PR00095 |
| | aminopeptidase | |
| | ras-related GTP binding | PR00098 |
| | protein | |
| | beta expansine EXPB1 | PR00104 |
| | Glycine rich protein | PR00105 |
| | metallothionein like protein | PR00108 |
| | (putative) | |
| | RCc3 strong root | PR00110 |
| weak discrimination center/ | uclacyanin 3-like protein | PR00111 |
| shoot meristem | | |
| very weak meristem | 26S proteasome regulatory | PR00116 |
| specific | particle non-ATPase | |
| | subunit 11 | |
| weak in endosperm | putative 40S ribosomal | PR00117 |
| | protein | |
| very weak in shoot | chlorophyll a/lo-binding | PR00122 |
| | protein precursor (Cab27) | |
| Strong leaves | putative | PR00123 |
| | protochlorophyllide | |
| | reductase | |
| strong discrimination center | metallothionein RiCMT | PR00126 |
| shoot meristem | | |
| Strong constitutive | GOS2 | PR00129 |
| | GOS9 | PR00131 |
| very weak meristem | chitinase Cht-3 | PR00133 |
| specific | | |
| Strong in endosperm | alpha-globulin | PR00135 |
| Weak in endosperm | alanine aminotransferase | PR00136 |
| | Cyclin A2 | PR00138 |
| | Cyclin D2 | PR00139 |
| | Cyclin D3 | PR00140 |
| Shoot and seed | Cyclophyllin 2 | PR00141 |
| medium constitutive | sucrose synthase SS1 | PR00146 |
| | (barley) | |
| weak in endosperm | trypsin inhibitor ITR1 | PR00147 |
| | (barley) | |
| strong constitutive | ubiquitine 2 with intron | PR00149 |
| Embryo and stress | WSI18 | PR00151 |
| | HVA22 homologue | PR00156 |
| | (putative) | |
| | EL2 | PR00157 |
| medium constitutive in | aquaporine | PR00169 |
| young plants | | |
| Strong constitutive | High mobility group | PR00170 |
| | protein | |
| weak constitutive | reversibly glycosylated | PR00171 |
| | protein RGP1 | |
| shoot | cytosolic MDH | PR00173 |
| Embryo and stress | RAB21 | PR00175 |
| | CDPK7 | PR00176 |
| very weak in meristem | Cdc2-1 | PR00177 |
| | sucrose synthase 3 | PR00197 |
| | OsVP1 | PRO0198 |
| very weak in young plant | OSH1 | PRO0200 |
| meristem | | |
| | putative chlorophyllase | PRO0208 |
| | OsNRT1 | PRO0210 |
| | EXP3 | PRO0211 |
| | phosphate transporter | PRO0216 |
| | OjPT1 | |
| aleurone + embryo | oleosin 18 kd | PRO0218 |
| | ubiquitine 2 without intron | PRO0219 |
| | RFL | PRO0220 |
| not detected | maize UBI delta intron | PRO0221 |
| | glutelin-1 | PRO0223 |
| | fragment of prolamin RP6 | PRO0224 |
| | promoter | |
| | 4xABRE | PRO0225 |
| | glutelin OSGLUA3 | PRO0226 |
| | BLZ-2_short (barley) | PRO0227 |
| | BLZ-2_long (barley) | PR00228 |

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

A particular enhancer element contemplated by the present invention is the tobacco etch virus translational enhancer (SEQ ID NO: 2).

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40. An exemplary sequence of a CaMV PolyA and terminator that may be used in the vector of the present invention is as set forth in SEQ ID NO: 5.

Other terminators contemplated by the present invention are thoses set forth in SEQ ID NO: 13, 19, 27, 33, 39, 45.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The vector of the present invention may comprise a nucleic acid sequence which encodes a polypeptide which confers antibiotic resistance. Such sequences are known in the art and include for examples those sequences set forth by SEQ ID NO: 3, the polypeptide sequence being as set forth in SEQ ID NO: 4.

The nucleic acid construct of the present invention may also comprise an additional nucleic acid sequence encoding a signal peptide that allows transport of the toxic peptide/chitinase in-frame fused thereto to a sub-cellular organelle within the plant, cell wall or secreted to the extra-cellular matrix, as desired. Examples of subcellular organelles of plant cells include, but are not limited to, leucoplasts, chloroplasts, chromoplasts, mitochondria, nuclei, peroxisomes, endoplasmic reticulum, apoplast and vacuoles.

Compartmentalization of the toxic peptide/chitinase recombinant protein within the plant cell followed by its secretion is one pre-requisite of making the product easily purifiable. It was shown that targeting a recombinant protein to the endoplasmic reticulum by fusion with an appropriate signal peptide allows the fused polypeptide to be targeted to a secretory pathway. Accumulation of the protein in a subcellular organelle of the cell may also be preferred to allow the protein to be stored in relatively high concentrations without being exposed to degrading compounds present in the vacuole, for example. Signaling sequences may be derived from plants such as wheat, barley, cotton, rice, soy, and potato.

Exemplary nucleic acid secretion signal sequences which direct polypeptides via the ER to the extracellular space include those set forth in SEQ ID NOs: 7 and 61-66. The amino acid sequence of this secretion signal sequence is set forth in SEQ ID NO: 8.

Another nucleic acid secretion signal sequence contemplated by the present invention is that set forth in SEQ ID NOs: 21, 67 and 68. The amino acid sequence of this secretion signal sequence is set forth in SEQ ID NO: 22.

Additional signal peptides that may be used herein include the tobacco pathogenesis related protein (PR-S) signal sequence (Sijmons et al., 1990, Bio/technology, 8:217-221), lectin signal sequence (Boehn et al., 2000, Transgenic Res, 9(6):477-86), signal sequence from the hydroxyproline-rich glycoprotein from Phaseolus vulgaris (Yan et al., 1997, Plant Phyiol. 115(3):915-24 and Corbin et al., 1987, Mol Cell Biol 7(12):4337-44), potato patatin signal sequence (Iturriaga, G et al., 1989, Plant Cell 1:381-390 and Bevan et al., 1986, Nuc. Acids Res. 41:4625-4638.) and the barley alpha amylase signal sequence (Rasmussen and Johansson, 1992, Plant Mol. Biol. 18(2):423-7). Such targeting signals may be cleaved in vivo from the toxic peptide/chitinase sequence, which is typically the case when an apoplast targeting signal, such as the tobacco pathogenesis related protein-S (PR-S) signal sequence (Sijmons et al., 1990, Bio/technology, 8:217-221) is used.

Other signal sequences which may also be used in accordance with this aspect of the present invention include signal retention sequences. Use of these sequences result in increased accumulation in a particular location and therefore may provide for easier purification of the toxic peptide/chitinase. For example, Pat. Appl. No. 20050039235 teaches the use of signal and retention polypeptides for targeting recombinant insulin to the ER or in an ER derived storage vesicle (e.g. an oil body) in plant cells thereby increasing the accumulation of insulin in seeds.

Examples of ER retention motifs include KDEL, HDEL, DDEL, ADEL and SDEL sequences.

As mentioned above, signal polypeptides may also be used for targeting the associated recombinant protein to the apoplast. It has been shown that targeting of recombinant immunoglobulins (MAb) to the apoplast significantly increased protein yields in comparison to plants where MAb was targeted to the cytosol [Conrad and Fiedler, 38 Plant Mol. Biol. 101-109 (1998)].

Yet another important strategy to facilitate purification/verification is to fuse the recombinant toxic peptide/chitinase with an affinity tag by including a sequence of the tag in the nucleic acid construct of the present invention. This method is widely utilized for in vitro purification of proteins. Exemplary purification tags for purposes of the invention include but are not limited to hemagglutinin epitope (HA TAG), polyhistidine, V5, myc, protein A, gluthatione-S-fransferase, maltose binding protein (MBP) and cellulose-binding domain (CBD) [Sassenfeld, 1990, TIBTECH, 8, 88-9]. The nucleic acid construct of the present invention may also comprise a sequence that aids in proteolytic cleavage, e.g., a thrombin cleavage sequence. Such a sequence may permit the toxic peptide/chitinase to be separated from an attached co-translated sequence such as the ER retention sequences described above.

Thus, the present invention encompasses nucleic acid sequences described hereinabove; fragments thereof, sequences hybridizable therewith, sequences homologous thereto, sequences orthologous thereto, sequences encoding similar polypeptides with different codon usage, altered sequences characterized by mutations, such as deletion, insertion or substitution of one or more nucleotides, either naturally occurring or man induced, either randomly or in a targeted fashion.

Plant cells may be transformed stably or transiently with the nucleic acid constructs of the present invention. In stable transformation, the nucleic acid molecule of the present invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by the present invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of the present invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsulated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of the present invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

Various construct schemes can be utilized to express two recombinant proteins from a single nucleic acid construct (i.e. the toxin and the chitinase). For example, the two recombinant proteins can be co-transcribed as a polycistronic message from a single promoter sequence of the nucleic acid construct. To enable co-translation of the toxin and the chitinase from a single polycistronic message, the first and second polynucleotide segments can be transcriptionally fused via a linker sequence including an internal ribosome entry site (IRES) sequence which enables the translation of the polynucleotide segment downstream of the IRES sequence. In this case, a transcribed polycistronic RNA molecule including the coding sequences of both the first and the second growth factors will be translated from both the capped 5′ end and the internal IRES sequence of the polycistronic RNA molecule to thereby produce both the toxin and the chitinase.

Alternatively, the first and second polynucleotide segments can be translationally fused via a protease recognition site cleavable by a protease expressed by the cell to be transformed with the nucleic acid construct. In this case, a chimeric polypeptide translated will be cleaved by the cell expressed protease to thereby generate both the toxin and the chitinase.

Still alternatively, the nucleic acid construct of the present invention can include two or more promoter sequences each being for separately expressing the toxin and the additional recombinant protein. These promoters which may be identical or distinct can be constitutive, tissue specific or regulatable (e.g. inducible) promoters functional in one or more cell types.

It will be appreciated that the toxic peptide and the chitinase may be expressed from two individual constructs (i.e. a nucleic acid construct system).

According to another aspect the toxic peptides of the present invention (and optionally the chitinase of the present invention) may be expressed in a heterologous system and provided to the insects as an insecticidal composition.

The host cells may be prokaryotic or eukaryotic such as bacterial, insect, fungal, plant or animal and in each case the regulatory sequences are adapted accordingly to enable expression of the polynucleotide(s) in the host species. For example, where the host cell is a plant cell, the regulatory sequence comprises a promoter active in plant cells, such promoters are well known to those skilled in the art and just one example is the promoter of the polyubiquitin gene of maize.

Optionally, the toxic peptides/chitinase may be used following recovery. The term “recovery” refers to at least a partial purification to yield a plant extract, homogenate, fraction of plant homogenate or the like. Partial purification may comprise, but is not limited to disrupting plant cellular structures thereby creating a composition comprising soluble plant components, and insoluble plant components which may be separated for example, but not limited to, by centrifugation, filtration or a combination thereof. In this regard, proteins secreted within the extracellular space of leaf or other tissues could be readily obtained using vacuum or centrifugal extraction, or tissues could be extracted under pressure by passage through rollers or grinding or the like to squeeze or liberate the protein free from within the extracellular space. Minimal recovery could also involve preparation of crude extracts of toxic peptides/chitinase, since these preparations would have negligible contamination from secondary plant products. Further, minimal recovery may involve methods such as those employed for the preparation of F1P as disclosed in Woodleif et al., Tobacco Sci. 25, 83-86 (1981). These methods include aqueous extraction of soluble protein from green tobacco leaves by precipitation with any suitable salt, for example but not limited to KHSO₄. Other methods may include large scale maceration and juice extraction in order to permit the direct use of the extract.

Alternatively, recovery of the toxic peptides/chitinase polypeptide from the plant (whole plant) or plant culture can be effected using more sophisticated purification methods which are well known in the art. For example, collection and/or purification of toxic peptides/chitinase from plant cells or plants can depend upon the particular expression system and the expressed sequence. Separation and purification techniques can include, for example, ultra filtration, affinity chromatography and or electrophoresis. In particular instances, molecular biological techniques known to those skilled in the art can be utilized to produce variants having one or more heterologous peptides which can assist in protein purification (purification tags, as described above). Such heterologous peptides can be retained in the final functional protein or can be removed during or subsequent to the collection/isolation/purification processing.

Thus, according to a further aspect of the invention there is provided an insecticidal composition comprising the aforementioned peptides/chitinases. Preferably the composition as hereinbefore defined is in the form of any desired formulation such as a solution, emulsion, spray, suspension, powder, foam, paste, granule, aerosol, capsule or other finely or coarsely divided material or impregnant for natural or synthetic material.

In one embodiment the insecticidal composition is in the form of a spray, suspension or the like, in admixture with suitable diluents, adjuvants, preservatives, dispersants, solvents, emulsifying agents or the like. Suitable composition components are those conventionally employed in the art, and in particular being suited to the present oral administration application. The composition may be obtained with use of any suitable solvents, preferably water, alcohol, mineral oil or the like, any suitable solid carriers such as kaolin, clay, talc, chalk, quartz, attapulgite, montmorillonite, diatomaceous earth, silica, or the like, with use of any solid carriers as supports for granules such as calcite, marble, pumice and crushed natural fibre material or the like. Compositions for use in the invention may additionally be employed in intimate or physical admixture together with other known insecticides, growth promoting or regulating substances, herbicides, fungicides, synergistic agents and the like.

The composition is preferably suitable for physically or chemically associating with plants or their locus, and for oral uptake by pathogens.

According to a preferred embodiment of the invention there is provided a method for the production of the aforementioned composition comprising: culturing the aforementioned host cell under conditions suitable for expression of the fusion protein; and harvesting the toxic peptides/chitinase from the culture.

According to a further aspect of the invention there is provided a method for the production of transgenic plant cells or plants that are resistant to disease comprising: transforming a selected plant genome with the aforementioned constructs(s) of the invention, as described herein above.

According to a yet further aspect of the invention there is provided a transgenic plant cell or plant, or their progeny, produced by the above method.

It is to be understood that the invention is not necessarily limited in its application to the details set forth in the following description. The invention is capable of other embodiments or of being practiced or carried out in various ways.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Construction of Vector 257

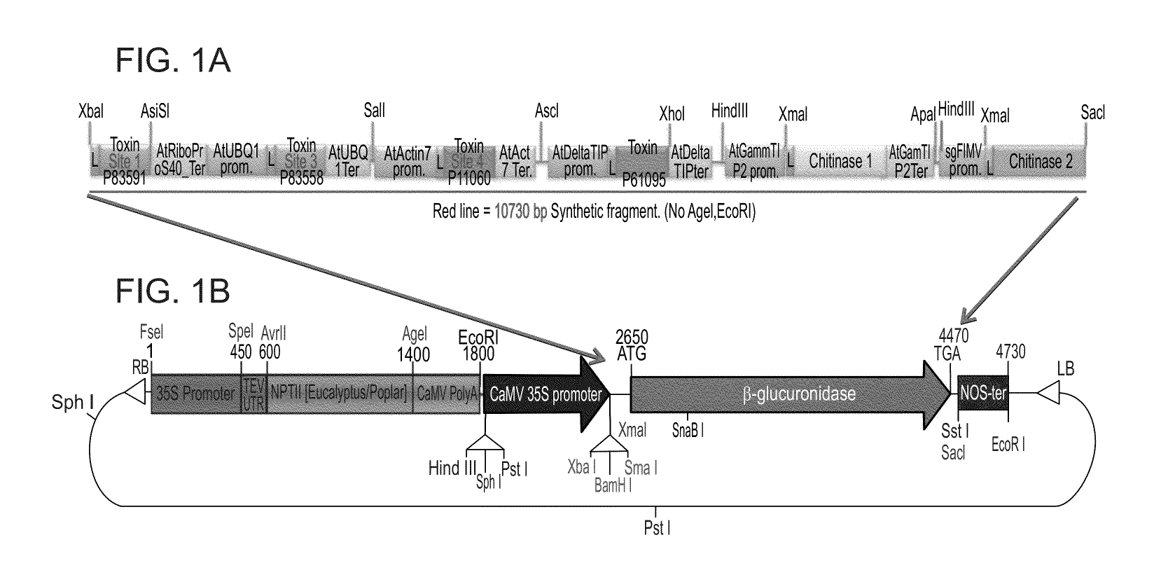

Construct 157 (altogether 14115 bp, as illustrated in FIG. 1B) which comprises the binary vector pBI121, GenBank: AF485783.1, a NPTII selective element (SEQ ID NO: 3), a 35S promoter (SEQ ID NO: 1) and a CaMV terminator (SEQ ID NO: 5) and glucoronidase was digested with Xba and Sac 1 and the AntiInsects6Cassete polynucleotide (10730 bp) was inserted into the construct as illustrated in FIGS. 1A-B.

In order to confirm that the AntiInsects6Cassete polynucleotide correctly inserted into the vector, restriction analysis was performed. Specifically, the DNA was co-digested with XbaI+SalI+SacI. The expected fragment size is: 12275 bp+8640 bp+2090 bp. Further, sequencing of all protein coding regions using standard methods was performed.

Results

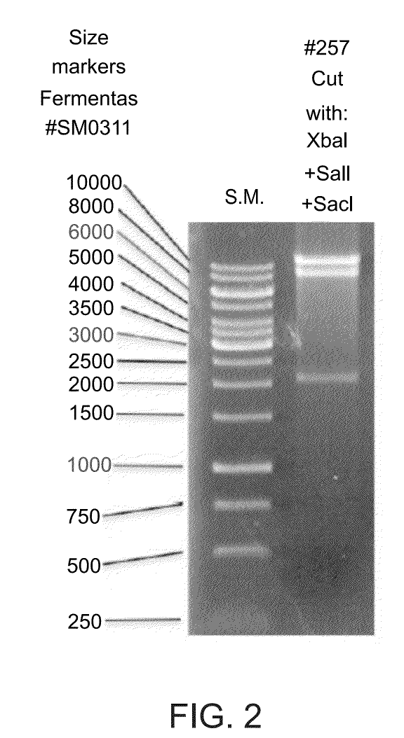

As illustrated in FIG. 2, vector 257 was confirmed to comprise the AntiInsects6Cassete polynucleotide.

### Example 2

### Construction of Vector 258

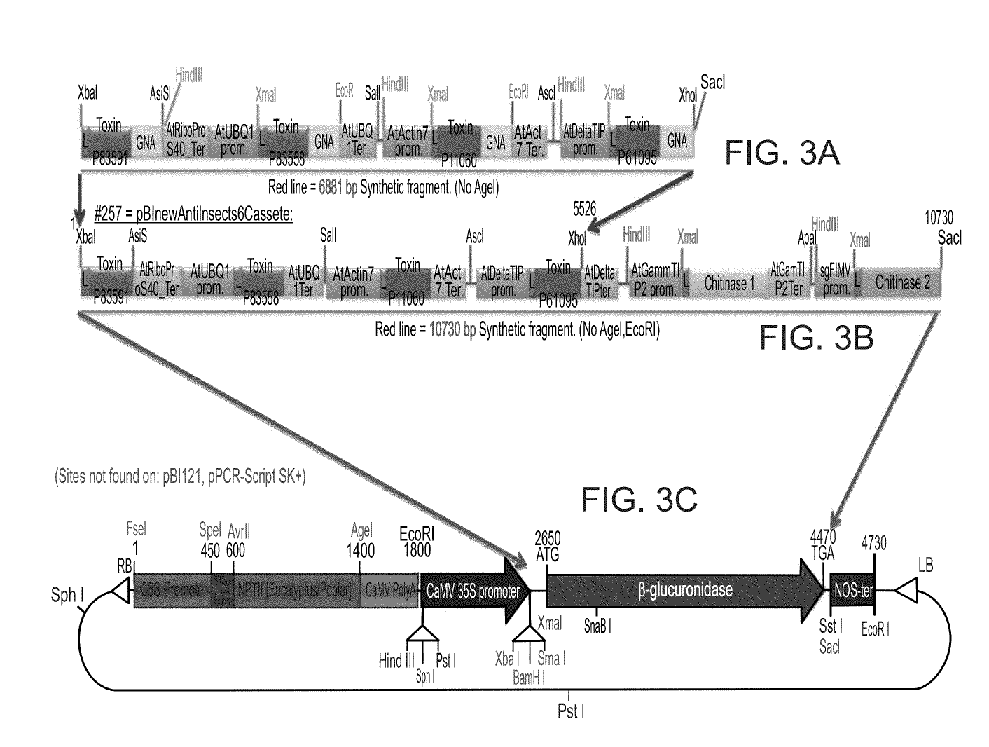

Vector #258 (14115 b. p.) was constructed by insertion of synthetic fragment AntiInsects6CDS_GNA (6881 bp) into the AntiInsects6Cassette polynucleotide using XbaI-XhoI, as illustrated in FIGS. 3A-B. The obtained insert was subsequently digested with Xba and SacI and inserted into vector 157 in order to obtain vector 258.

In order to confirm that the AntiInsects6CDS GNA polynucleotide correctly inserted into the vector, restriction analysis was performed. Specifically, the DNA was co-digested with XbaI+XhoI. The expected fragment size is: 17499 bp+6881. Further, sequencing of all protein coding regions using standard methods was performed.

Results

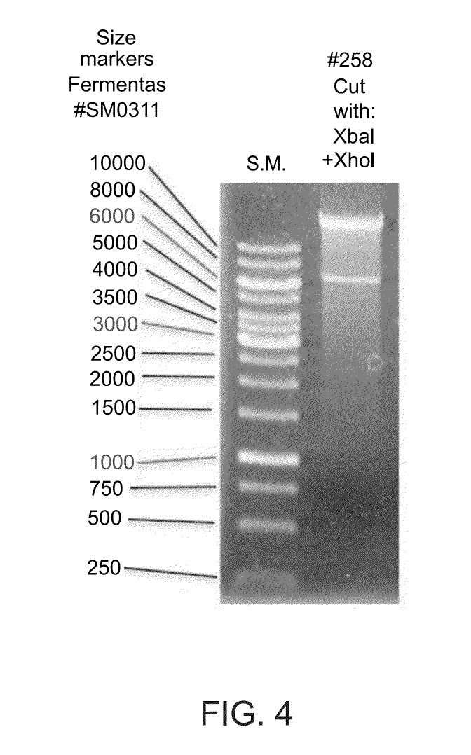

As illustrated in FIG. 4, vector 258 was confirmed to comprise the AntiInsects6CDS GNA polynucleotide.

### Example 3

### Transformation of Tobacco with Vectors 257 and 258

Materials and Methods Mediums:

TR: 4.4 gr/L of MS salts+vitamins (Duchefa. Cat#M0222), 3% Sucrose 30 gr/lLiter), adjusted to pH 5.8 by KOH. 0.65% Plant Agar (3.25 gr/0.5L added separately to each bottle), autoclave.

Liquid TR: 4.4 gr/L of MS salts+vitamins (Duchefa. Cat#M0222), 3% Sucrose (30 gr/1 Liter), adjusted to pH 5.8 by KOH.

TR+H: TR with the additional hormones: 2 mg/L Zeatin+0.1 mg/L IAA; 2 mg/L Kinetin+0.8 mg/L IAA.

Preparation of agrobacterium: Agrobacterium culture was grown in 100 ml LB+Rifampicin (100 mg/L), and 50 mg/L of kanamycin at 28° C., 200 rpm for 48 hours.

Preparation of tobacco explants: Fresh N. tabacum plants with dark green leaves were taken. The leaves were cut into ˜1 cm×1 cm pieces and placed on 140 mm Petri dish containing TR liquid medium (p.H 5.8).

Eliminating traces of antibiotics: Once the agrobacterium culture reached an OD of 1-2.0 (about 24-48 hours), 30 ml of the culture was spun down and the pellet was re-suspended in 30 ml of TR liquid medium (p.H 5.8). The suspended culture was spun down again and resuspended in TR to a final concentration of OD₆₀₀.=0.5-1.0.

Co-cultivation: The TR liquid medium (see preparation of tobacco explants) was removed from the plate and Agro suspension was added instead. Using a scalpe,l two cuts were made in the main vessel of each leaf. The leaves were incubated in the agro suspension for 5-30 minutes. The agro suspension was removed from the plate and the leaves dry-blotted on sterile paper. Explants were transferred to solid TR+H plates, with the upper side of the leaf facing up. Co-cultivation was effected for 2 days.

Selection/regeneration: 5/6 explants per plate were transferred to TR+H+ selection plates. Selection medium included antibiotics to eliminate agro growth (e.g. Cefatoxime (200 mg/L) and Carbenicillin (320 mg/L) or Augmentin (200 mg/L) or Timentin (100 mg/L) together with relevant antibiotic for selecting the transgenic shoots (Kana 100 mg/L /hygromycin 25 mg/L). It was important that the explants had full contact with the medium. The selection medium was replaced every 7 days (Augmentin and Tlmentin, degraded after a week), until shoots developed.

Rooting: By six weeks, single shoots were transferred to TR medium containing selection antibiotics.

Verification: In order to verify that the transgene is expressed in the plant, Western blot analysis with NPTII antibodies may be performed. Further PCR analysis and/or RT-PCR analysis with NPTII primers, chitinase 1 primers and toxin primers may be performed

### Example 4

### Transformation of Eucalyptus hypocotyls and Cotyledon with Vectors 257 and 258

Plant material: Seeds of E. tereticomis were surface-disinfected with 70% ethanol for 2 minutes and 0.1% (w/v) aqueous mercuric chloride solution for 10 min and washed with sterilized distilled water three times.

Twenty seeds per plate were germinated aseptically in 90×15-mm Petri dishes containing 25 ml of seed germination medium composed of the MS basal medium consisting of 3% (w/v) sucrose and 0.8% (w/v) agar.

Agrobacterium: LBA 4404 strain of A. tumefaciens harboring vector 257 or 258 was used for transformation. Bacterial culture collected at late log phase (A600) were pelleted and resuspended in MS basal medium.

Protocol: Cotyledon and hypocotyls explants from 7-d-old seedlings were separated and used as explants for transformation experiments.

The explants were precultured on the MS regeneration medium supplemented with 0.5 mg/l BAP and 0.1 mg/l NAA for 2 d.

The precultured cotyledon and hypocotyl explants were gently shaken in the bacterial suspension for 10 minutes and blotted dry on a sterile filter paper. Afterwards, they were transferred to MS regeneration medium supplemented with 0.5 mg/l BAP and 0.1 mg/l NAA for 2 days.

Following co-cultivation, the explants were washed in the MS liquid medium, blotted dry on a sterile filter paper, and transferred to MS regeneration medium containing 0.5 mg/l BAP and 0.1 mg/l NAA supplemented with 40 mg/l kanamycin and 300 mg/l cefotaxime.

Following 4-5 wk of culture, regeneration was observed from the edges of explants. The explants were transferred to liquid elongation medium (MS medium supplemented with 0.5 mg/l BAP, 40 mg/l kanamycin, and 300 mg/l cefotaxime) on paper bridges.

The elongated shoots (1.5-2 cm) were rooted in the MS medium with 1.0 mg/l IBA and 40 mg/l kanamycin.

Verification: In order to verify that the transgene is expressed in the plant, Western blot analysis with NPTII antibodies may be performed. Further PCR analysis and/or RT-PCR analysis with NPTII primers, chitinase 1 primers and toxin primers may be performed

### EXAMPLE 5

### Assaying for Expression of Toxic Peptides in the Eucalyptus Plants

Host plant: Eucalyptus camaldulensis clone 118.

Target organism: 1. Gall wasp Leptocibe invasa, 2. Gall wasp Ophelimus maskelli.

E. camaldulensis is transformed with vector 257, vector 258 or with vector alone for control. Transgenic, wt and control eucalyptus plants are grown in insect proof cages in the greenhouse together with adult gall wasps. The insect proof cages keep the inoculums in, while preventing outside pests from entering the cage. Following wasp inoculation, the appearance of galls in the veins and in the leaves is evaluated. Plants are examined to determine number of galls, gall size (maximum length), number of vital larvae in galls and number of emerging matured gall wasps. Five independent transformation events of transgenic eucalyptus are tested. Ten lines of each transformation event are inoculated with adult gall wasps in 3 independent repeats. Number of galls, gall size, vital larvae per 10 galls and emerging adults (by the exit hole) are recorded 1, 2, 3 and 4 months after inoculation.

### Example 6

### Assaying for Expression of Toxic Peptides in the Tobacco Plants

Host Plant: Tobacco.

Target organism: Whitefly Bemisia tabaci.

Nicotiana tabaccum is transformed with vector 257, 258 or with vector alone for control.

Transgenic, wt and control tobacco plants are grown in insect proof green house. 3 repeats of each best expressing plant lines are placed in insect proof cages. 100 moult-synchronized B. tabaci will be collected in a special container built around one leaf (3 leaves per plant—total of 81 special containers). Every 3 days, the surviving B. tabaci are counted to calculate percentage mortality.

### Example 7

### Assaying for Expression of Toxic Peptides in the Tobacco Plants

Host Plant: Tobacco.

Target organism: Lepidoptera, Spodoptera littoralis.

Nicotiana tabaccum transformed with vectors 257 or 258 or with vector alone for control.

Transgenic, wt and control tobacco plants are grown in insect proof green house. 3 repeats of each best expressing plant lines are placed in insect proof cages. 30 moult-synchronized S. littoralis are placed in a petri dish with one leaf (3 leaves per plant—total of 81 special containers). Every day, the surviving S. littoralis are counted to calculate percentage mortality. Every day, the damage for the eaten leaf is recorded and compared to the wt and control leaves. This bioassay is repeated for molt level #1, #3 and #5.

Results

Transgenic plants transcribing the spider toxin and chitinase construct are expected to exhibit significantly higher S. littoralis mortality and invisible leaf eating damage. Transgenic plants lines are resistant to S. littoralis infection, compared to control and wt plants that are infected, fully eaten and cause no S. littoralis death.

1. An isolated polynucleotide comprising:
(i) a nucleic acid sequence encoding at least one toxic peptide, said toxic peptide being a spider toxin; and
(ii) a nucleic acid sequence encoding a chitinase attached to a secretion signal sequence.

2. An isolated polynucleotide comprising:
(i) a nucleic acid sequence encoding at least one toxic peptide, said toxic peptide not being chitinase; and
(ii) a nucleic acid sequence encoding a chitinase.

3. The isolated polynucleotide of claim 2, wherein said toxic peptide is derived from insects selected from the group consisting of bees, wasps, cockroach, blowfly, mosquito, webworm, beetle, antipode, millipede, crab, lobster, shrimp, prawn, spider, scorpion, mite and tick.

4. The isolated polynucleotide of claim 2, wherein said toxic peptide is derived from a spider.

5. The isolated polynucleotide of claims 1, wherein said toxic peptide comprises an amino acid sequence at least 90% homologous, and/or at least 80% identical to a sequence selected from the group consisting of SEQ ID NOs: 9, 15, 24, 30, 55, 56 and 57 as determined using the BlastP software of the National Center of Biotechnology Information (NCBI) using default parameters.

6. The isolated polynucleotide of claim 1, wherein said toxic peptide is attached to a plant lectin.

7. The isolated polynucleotide of claim 1, wherein said chitinase is not attached to a plant lectin.

8. The isolated polynucleotide of claim 6, wherein said plant lectin comprises Galanthus nivalis agglutinin (GNA).

9. The isolated polynucleotide of claim 1, wherein said toxic peptide is attached to a secretion signal sequence.

10. The isolated polynucleotide of claim 2, wherein said chitinase is attached to a secretion signal sequence.

11. The isolated polynucleotide of claim 1, wherein said secretion signal sequence is encoded by a nucleic acid as set forth in SEQ ID NOs: 7, 21 and 61-68.

12. The isolated polynucleotide of claim 1, wherein said chitinase comprises an amino acid sequence at least 90% homologous, and/or at least 80% identical to a sequence selected from the group consisting of SEQ ID NO: 36, 42 and 58-60, as determined using the BlastP software of the National Center of Biotechnology Information (NCBI) using default parameters.

13. The isolated polynucleotide of claim 1, wherein said at least one toxic peptide comprises a first and a second toxic peptide, wherein said first toxic peptide targets a first site in a sodium (Naᵥ) channel and said second toxic peptide targets a second site in said Naᵥ channel.

14. The isolated polynucleotide of claim 1, wherein said at least one toxic peptide comprises a first, second and a third toxic peptide, wherein said first toxic peptide targets a first site in a sodium (Naᵥ) channel, said second toxic peptide targets a second site in said Naᵥ channel and said third toxic peptide targets a third site in said Naᵥ channel.

15. The isolated polynucleotide of claim 14, wherein said first toxic peptide is P83591, said second toxic peptide is P83558 and said third toxic peptide is P11060.

16. The isolated polynucleotide of claim 1, wherein said at least one toxic peptide comprises at least 4 toxic peptides, wherein a first of said at least 4 toxic peptides is P83591, a second of said at least 4 toxic peptides is P83558, a third of said at least 4 toxic peptides is P11060, a fourth of said at least 4 toxic peptides is P61095.

17. A nucleic acid construct comprising the isolated polynucleotide of claim 1 and a cis regulatory element.

18. A nucleic acid construct system comprising:
(i) a first nucleic acid construct comprising an isolated polynucleotide which comprises a nucleic acid sequence encoding a toxic peptide and a cis regulatory element; and
(ii) a second nucleic acid construct comprising an isolated polynucleotide which comprises a nucleic acid sequence encoding a chitinase and a cis regulatory element.

19. The nucleic acid construct system of claim 18, wherein said toxic peptide is attached to a plant lectin or a secretion signal sequence.

20. (canceled)

21. The nucleic acid construct system of claim 18, wherein said chitinase is attached to a secretion signal sequence.

22-25. (canceled)

26. A nucleic acid construct comprising the isolated polynucleotide of claim 5 and a cis regulatory element.

27. A nucleic acid construct system comprising:
(i) the nucleic acid construct of claim 26; and
(ii) a nucleic acid construct comprising an isolated polynucleotide which comprises a nucleic acid sequence encoding a chitinase.

28-31. (canceled)

32. A plant comprising the nucleic acid construct of claim 17.

33. A plant comprising the nucleic acid construct system of claim 18.

34-35. (canceled)

36. An insecticidal composition comprising:
(i) a toxic peptide; and
(ii) a chitinase.

37. (canceled)

38. A method of controlling or exterminating an insect, the method comprising expressing in a host plant of said insect the isolated polynucleotides of claim 1, thereby controlling or exterminating the insect.

39. A method of controlling or exterminating an insect, the method comprising expressing in a host plant of said insect an isolated polynucleotide which comprises a nucleic acid sequence encoding a toxic peptide selected from the group consisting of SEQ ID NOs: 9, 15, 24, 30 and 55-57.

40. A method of controlling or exterminating an insect, the method comprising contacting the insect with the insecticidal compositions of claim 36, thereby controlling or exterminating the insect.

41-45. (canceled)
